(19) United States
(12) Patent Application Publication
(10) Pub. No.: US 2006/0128657 A1
(43) Pub. Date: Jun. 15, 2006

(54) Selected betaines and their uses

(76) Inventor: Jallal Messadek, Liege (BE)

Correspondence Address: HOVEY WILLIAMS LLP, Suite 400, 2405 Grand, Kansas City, MO, 64108, US

(21) Appl. No.: 11/348,142

(22) Filed: Feb. 6, 2006

Related U.S. Application Data
(63) Continuation-in-part of application No. 10/635,048, filed on Aug. 4, 2003.
(63) Continuation-in-part of application No. PCT/BE04/00110, filed on Aug. 3, 2004.
(63) Continuation-in-part of application No. PCT/BE04/00043, filed on Mar. 23, 2004.

(51) Int. Cl. A61K 31/727; A61K 31/205

(52) U.S. Cl. 514/56; 514/554

(57) ABSTRACT

A physiologically acceptable, sterile and pyrogen-free solution of betaine dissolved in a physiologically acceptable solvent, having a pH adjusted to from 5.0 to 8.0 with a betaine concentration of from 5 to 500 mg/ml.

The present application is a continuation-in-part application of U.S. application Ser. No. 10/635,048 filed on Aug. 4, 2003, PCT/BE 2004/000110 filed on Aug. 3, 2004, and PCT/BE 2004/000043 filed Mar. 23, 2004, the contents of which are incorporated herein by reference.

### BACKGROUND OF THE INVENTION

1. Field of the Invention

According to the invention there is provided a sterile, pyrogen-free, preferably ready-to-use solution or composition of a betaine, which consists essentially of a physiologically acceptable betaine or a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof and mixtures thereof dissolved in a physiologically acceptable solvent thereof, which has a pH of from 5 to 8. The solution of the invention is particularly advantageous for the administration by injection of the betaine drugs, in the treatment of both human and animal blood troubles, especially coagulation troubles and bleeding troubles.

2. Prior Art

The use of injectable glycine betaine composition has been proposed in co-pending US application US 2004/0033223 filed on Aug. 4, 2003 and PCT/BE 2004/000043 filed Mar. 23, 2004, the content of which are incorporated by reference.

It has been observed that when subjecting ready to use glycine betaine solution to sterilisation step, such as heat treatment step at more than 121° C. for more than 30 minutes, the efficiency of the glycine betaine solution was for specific lots diminished as observed in tests in vivo and in vitro. It was thus of interest to determine in the betaines solutions being submitted to a sterilisation process those retaining their pharmacological properties.

The present invention has for subject matter a simple method for determining whether a glycine betaine composition or solution has a very good efficiency for treating side effect of heparin, low molecular heparin, heparin like molecules, especially Arixtra® and Lovenox®. The invention relates thus also to a glycine betaine composition having a high efficiency for treating side effects of new molecules and treatments such as Lovenox®, Fraxiparine®, Fragmin®, Arixtra®, Exanta®, Angiomax® and Refludan®.

### BRIEF DESCRIPTION OF THE INVENTION

The invention relates to a betaine sterile and pyrogen-free physiologically acceptable pharmaceutical injectable composition having a pH adjusted to from 5.0 to 8.0 with a betaine concentration from 0.1 to 1000 mg/ml, wherein said pharmaceutical composition containing a betaine and having a betaine pharmacological activity characterized in that when combining 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined aqueous solutions have a spectrometric absorbance of at least 0.9 advantageously at least 0.95, preferably at least 1.0, more preferably at least 1.1, more specifically more than 1.2, specifically more than 1.3 at a temperature of 20° C., at 632 nm wave length after been mixed at a temperature of 20° C.

According to a preferred embodiment, the betaine sterile and pyrogen-free physiologically acceptable pharmaceutical injectable composition has a pH adjusted to from 5.0 to 8.0 with a betaine concentration of from 0.1 to 500 mg/ml, wherein said solution is optionally enriched in sodium, magnesium or potassium, whereby the composition has such an activity that the betaine composition mixed with heparin and thereafter with Azure A and water so as to achieve a composition with a spectrometric absorbance at 632 nm wave length of at least 0.9, advantageously at least 0.95, preferably at least 1, more preferably at least 1.1, more specifically more than 1.2, specifically more than 1.3, at a temperature of 20° C.

Advantageously, the composition is substantially free of dimethyl glycine and/or substantially free of sarcosine and/or substantially free of glycine. Advantageously, the composition is substantially free of degradations products subsequent to a sterilisation process. In one embodiment the betaine starting solution can be, for example, calculated at 110% of betaine weight as to reach 100% in case of 10% betaine degradation, if any, during the sterilisation process. Of course the added percentage can be adjusted depending of betaine possible degradation during heating, gamma rays, electron and/or sterilisation processes.

Advantageously, the composition is substantially free of degradations products subsequent to a heating, gamma or electron sterilisation process.

Advantageously, the composition is substantially free of degradations products such as toxins and pyrogens issued from micro-organisms, such composition having bioburden values acceptable by the International Pharmacopoeia.

Preferably, the composition is enriched in sodium, magnesium or potassium.

Preferably, at least 95% by weight, preferably at least 99% by weight of the solution or composition consists of:

physiologically acceptable betaine or a physiologically acceptable salt thereof;

one or more physiologically acceptable salts of sodium, magnesium, potassium or a mixture thereof; and

physiologically acceptable solvent

The solution or composition has advantageously an osmolality comprised between 200 and 500 mOsm/kg, preferably between 270 and 350 mOsm/kg and a viscosity comprised between 0.5 and 50 m Pa·s, preferably between 1-10 m Pa·s.

The solution or composition comprises advantageously one or more salts of sodium, magnesium and potassium selected from the group consisting of chloride, hydroxide, sulfate and mixtures thereof.

The weight ratio betaine or salt thereof/physiologically acceptable salts selected from sodium, magnesium, potassium and mixture thereof is advantageously greater than 3, preferably comprised between 5 and 100, such as 10, 15, 20, 25, 30, 35 40, 45, 50, 60, 70, 80, 90, 100, betaine in such ratios being the major compound by weight in the solvent.

The solution or composition is preferably contained in a sealed container, advantageously before its sterilisation, possibly after one or more filtration step. According to an embodiment, the betaine solution or composition is submitted to a filtration step before being filled and sealed in sterilized container(s).

Advantageously, the sealed container has a layer in contact with the composition which is substantially free of Si atoms and/or which is substantially free of N atoms.

Preferably, the sealed container has a layer in contact with the composition which is made of a synthetic material selected from the group consisting of polyethylene, polypropylene, copolymers of ethylene and propylene, polycarbonate, and mixtures thereof.

Most preferably, the sealed container has a layer forming a barrier to the light.

According to a specific embodiment, the solution or composition is contained in a sealed container with a free volume corresponding to less than 10% of the volume of solution, preferably to less than 5% of the volume solution.

The solution or composition has advantageously been submitted to a filtration with an absolute filter of less than 1 μm.

For example, the solution or composition has been submitted to a filtration with a filter lower than 0.3 μm, preferably lower than 0.22 μm, such as a filter 0.1 μm, most preferably equal or lower than 0.01 μm.

Advantageously, the solution is contained in a sealed container having one or more inner surfaces not in contact with the solution, whereby said inner surface or surfaces are substantially free from salt deposits.

The physiologically acceptable solvent is for example selected from the group consisting of water, ethanol, polyethylene glycol, dimethylacetamide, aqueous polyvinylpyrrolidone, propylene glycol and mixtures thereof. Water is however preferred.

Said water contains advantageously less than 100 ppm salts.

According to a specific embodiment, at least 95% by weight, preferably at least 99% by weight of the solution consists of:

physiologically acceptable betaine or a physiologically acceptable salt thereof;

one or more physiologically acceptable salts of only one element selected from the group consisting of sodium, magnesium and potassium; and

physiologically acceptable solvent

The concentration of betaine is for example from 10 to 700 mg/ml, preferably from 30 to 300 mg/ml.

The solution or composition can further comprise a tonicity adjusting agent.

The invention relates also to a process for the preparation of a solution according to anyone of the preceding claims, which comprises at least the following steps:

preparation of a solution comprising betaine or a salt thereof, said solution being enriched in sodium or magnesium or potassium,

filtration of the solution,

filling of vials,

sealing of the filled vials, and

sterilisation of the sealed vials,

whereby during or just after the sterilization step, the sterilized sealed vials are shacked or submitted to a step suitable for mixing the content of the vial.

In one embodiment of the invention, a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof, will be used as unique compound to prepare the ready to use or the reconstituted injectable (e.g. parenteral, subcutaneous and intravenous) solutions. In effect, it has been discovered that a pure glycine betaine solution in pyrogen free injectable water possesses improved biological activity while being more stable than solutions with additives. Unexpectedly the applicant discovered that when dissolving a pure amount of a betaine in pyrogen free water of injection, without adding any additive such as solvents, salts, acids, bases, preservatives or any compound generally added to an injectable drug, it was possible to obtain iso-osmotic solutions with an acceptable pH, i.e. physiologically acceptable injectable solutions, such solutions been ready to use in a patient in need or ready to be reconstituted as physiologically acceptable injectable solutions solubilized in physiologically acceptable injectable medias and/or solvents. Such pure betaine injectable solution showed improved pharmacological efficacy comparatively to betaine injectable solutions supplemented with additives.

In one embodiment higher concentrations of at least a betaine can be used to provide ready to use stock solutions, said stock solutions been used to reconstitute injectable solutions with an acceptable osmolality, such as 250 to 1450 mOsm/kg, preferably between 250 and 650 mOsm/kg and/or an acceptable pH between 5 and 8. The solvent used for reconstituting injectable physiological solutions from the stock solution can be pyrogen free injectable water or any solvent known by the skilled man.

Injectable pure betaines solutions showed better stability and conservation than those with additives. Unexpectedly, when submitted to autoclaving, betaine pure solutions showed more stability and less degradation than betaines solutions where additives were present.

In one embodiment higher concentrations of at least a betaine can be used to provide ready to use stock solutions, said stock solutions been used to reconstitute injectable solutions (e.g. parenteral, subcutaneous, intramuscular and intravenous) with an acceptable osmolality, such as 250 to 1450 mOsm/kg, preferably between 250 and 650 mOsm/kg, and/or an acceptable pH between 5 and 8, said stock solution being during the manufacturing process submitted to a microfiltration, an ultrafiltration, a nanofiltration or an osmosis reverse. The stock solutions can be reconstituted in physiological water and/or in ultra pure pyrogen free water or in any physiological solvent.

In one embodiment during the manufacturing process the pyrogen free betaines solutions are submitted to an ultrafiltration, a nanofiltration or an osmosis reverse before being lyophilised. The obtained lyophilised betaines compounds can be pyrogens, endotoxins, pesticides, herbicides and heavy metals free and will be hermetically sealed in aseptic containers. Such containers are suitable for reconstituting, preferably in aseptic manner, in pyrogen free water or in any physiological solvent, the injectable solutions of the invention.

In one embodiment, the aim of the present invention is to provide a pharmaceutical injectable betaine solution which is substantially free of any contamination or degradation product. For this purpose all the known techniques of filtration, autoclaving, pasteurisation or sterilisation can be used.

The filtration techniques particularly suitable for obtaining the pharmaceutical injectable pure betaine solutions of the invention are the microfiltration, the ultrafiltration, the nanofiltration and the osmosis reverse. These filtration steps as a nanofiltration process is characterized in that such nanofiltration is carried out using a nanofiltration membrane selected from polymeric and inorganic membranes having a cut-off size of 50 to 2500 Angstroms. The nanofiltration membrane is selected from hydrophobic membranes and/or hydrophilic membranes and/or ionic membranes. The obtained retentate and/or permeate of the filtration process may be utilized to realize the pharmaceutical pure betaine solutions of the invention.

In one embodiment, the obtained retentate and/or permeate of the filtration process are optionally submitted to an autoclaving process and/or pasteurisation process and/or sterilisation process before further steps as conditioning in sealed containers.

In one embodiment, the pharmaceutical pure betaine solutions are submitted to an autoclaving process and/or pasteurisation process and/or sterilisation process before further steps as a filtration process and/or microfiltration process and/or ultrafiltration process and/or nanofiltration and/or osmosis reverse process, and/or their combinations.

In one embodiment, the obtained retentate and/or permeate of the filtration process are lyophilized and hermetically sealed in aseptic containers. Such containers been suitable for reconstituting, preferably in aseptic manner, in pyrogen free water the injectable solutions of the invention.

In one embodiment, instead of autoclaving the injectable solutions of the invention at a temperature of 121° C. during 30 minutes a longer cycle of autoclave can be used, such longer autoclaving permitting to lower the autoclave temperatures from 121° C. to 110° C. or to 101° C. for example. The adapted longer periods of autoclave will allow attaining specific bioburden values, such as those required by the International Pharmacopoeia for parenteral drugs (intravenous), while preserving the pharmacological activities of the compositions of the invention. Such lower autoclaving temperatures can avoid the degradation of the injectable solutions of the invention, retaining their physical, biological and pharmacological properties. The injectable ready to use or reconstituted solutions can be adjusted for different osmolarities and/or pH and/or viscosity and/or any physical property, depending of the therapeutically purpose i.e. as for bleeding antidote purposes, antithrombotic purposes, anti-inflammatory purposes, anti-cancerous purposes, other drugs carrying purposes, other drugs delivering purposes, other drugs releasing purposes or other drugs potentialising therapeutically effects purposes.

According to a particularly preferred feature of the invention, there is provided a sterile, pyrogen-free, betaine solution which consists essentially of a physiologically acceptable betaine or salt thereof dissolved in a physiologically acceptable solvent thereof, which has a pH of from 5 to 8 and osmolality preferably comprised between 250 and 1450 mOsm/kg, such betaine solution been further submitted to a sterilisation process. The principle of such pure betaines solutions rests in the fact that therapeutically effective doses of a betaine can be attained without adding any additive or excipient in the injectable solutions. Betaine physical properties allow having physiological solutions with optimal betaines concentrations, betaine serving for its own for attaining physiological balance, i.e. osmolality, pH, etc. Preferably concentrations since 30 mg/ml to 300 mg/ml can be used.

In one embodiment, the pure betaine solution by avoiding in their manufacture process the handling of different ingredients such as those known by the skilled man for stabilising, preserving or balancing (osmolality, pH, etc), render their manufacture process more simple with less possibilities for contaminations, as for example those which can arise during a mixing process or from the contaminants inside the added products. By avoiding other ingredients than betaines, the solutions of the invention limit also other contaminations which can arise with the degradations of these added ingredients. Additives and excipients augment the injectable solutions complexity rendering them less stable.

Betaine having shown to be very stable, the pure betaine solutions of the invention are expected to show the same stability and resistance to temperature.

In one embodiment, the betaine and/or the solvent can be submitted to a sterilisation process before been mixed and before to be submitted or not to a further sterilisation process. In one embodiment, the pharmaceutical pure betaine solutions of the invention can be submitted to a microfiltration, an ultrafiltration, a nanofiltration and/or osmosis reverse process. These filtration steps as a nanofiltration process is characterized in that such nanofiltration is carried out using a nanofiltration membrane selected from polymeric and inorganic membranes having a cut-off size of 50 to 2500 Angstroms.

The pure betaines solutions are characterized by a very good stability, not necessitating the addition of preservatives. Solutions in various solvents, but preferably in ultra pure pyrogen free water and with different pH and concentrations have been found to be stable for long periods at temperatures accepted for the storage of pharmaceutical preparations.

The pure betaines solutions, preferably glycine betaine from plant source as sugar beet, are also characterized by very good pharmacological activities.

In one embodiment of the invention the glycine betaine used in the pharmaceutical injectable solutions of the invention is obtained from sugar beet, advantageously from genetically modified sugar beet. Such transgenic sugar beets containing modified genes or DNA as to be environment stress/aggressions resistant. In one embodiment of the invention the glycine betaine used in the injectable solutions of the invention is obtained from sugar beet genetically modified or not, said sugar beet been modified as to be environment resistant and/or to have an improved endogen betaine production.

In one embodiment of the invention the glycine betaine used in the injectable solutions and/or the pharmaceutical compositions of the invention such as oral or (trans)dermal are obtained from sugar beet genetically modified or not said sugar beet been obtained by biological cultivation, i.e. free of herbicides and pesticides.

In one embodiment of the invention the glycine betaine used in the injectable solutions or compositions of the invention is obtained from sugar beet genetically modified or not said sugar beet been obtained by hydroponics cultivation.

In one embodiment of the invention the glycine betaine used in the injectable solutions of the invention is obtained from cell cultures of sugar beet genetically modified or not, said cells being submitted or not to a step of elicitation.

In one embodiment of the invention the glycine betaine used in the pharmaceutical injectable solutions of the invention can be obtained from other plant sources such as wheat germs and spinach. The extraction techniques/origins claimed above for beets can also be applied to these two plant sources of betaine.

In one embodiment of the invention it is claimed that the glycine betaine obtained from beets, wheat germs and spinach as described above can be used for various pharmaceutical purposes, notably for drug manufacture. Such obtained betaines being substantially free of contaminants of various origins.

### DESCRIPTION OF EMBODIMENTS

The present invention relates to stable intravenously injectable ready-to-use solutions of betaine, to processes for preparing such solutions, and provide the same in a sealed container, and to a method for treating blood disturbances by the use of the said ready-to-use solution. Such preparations are also suitable for subcutaneous, intravenous and/or intramuscular administrations.

The betaine is a well known compound having various activities, especially anti thrombotic activity and properties for lowering side effects of anti thrombotic agents different from betaine.

For different anti thrombotic compounds, such as heparin and heparin like compounds as ultra low molecular heparins such as oligosaccharides and pentasacharides such as fondaparinux sodium (Arixtra®), Idraparinux sodium, investigational anti-Xa agents such as DX 9065a, it is necessary to have at his disposal an antidote for annealing possible bleeding side effects. Said antidote heeds to be ready-to-use, so as to avoid the loss of time for injection of the antidote treatment. Betaine for these new anticoagulants such as factors Xa inhibitors showed unexpected properties to reverse their anticoagulant potency.

In one embodiment betaine can be used to reverse anticoagulation of molecules or drugs which target, antagonize, bind or inhibit other factors of anticoagulation than factor Xa.

In one embodiment betaine can be used to reverse anticoagulation of molecules or drugs which target, antagonize, bind or inhibit other factors of anticoagulation than factor Xa such as other approved anti-IIa agents such as argatroban, ximelagatran (Exanta®), melagatran, hirulog, lepirudin recombinant hirudin, bivalirudin as Angiomax® and others direct thrombin inhibitors as hirudin, bivalirudin, argatroban, efegatran, or inogatran.

Betaine in a general manner will bind to sulphated negatively charged pool of anticoagulants and glycosaminoglycans as demonstrated in Azure A binding tests.

Low molecular weight heparins (LMWH) are composed of sulfated polysaccharides, which provide an attractive binding target for compounds of general formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof.

The invention relates thus to a composition or a pharmaceutical combination comprising a therapeutic effective amount of a therapeutically active agent selected from:

A) one or more of the following compounds such as, heparin and heparin like compounds, synthetic heparins, synthetic heparin like compounds, low molecular heparins, ultra low molecular heparins such as oligosaccharides and pentasacharides such as fondaparinux sodium (Arixtra®), Idraparinux sodium, experimental ultra low molecular heparins, directs and indirects anti-Xa agents such as DX 9065a, anti factor IX agents, anti factor VII agents, directs and indirects anti coagulation factor agents, anti-IIa agents such as argatroban, ximelagatran (Exanta®), melagatran, lepirudin recombinant hirudin and hirulog as and others direct thrombin inhibitors as hirudin, bivalirudin (such as Angiomax®), argatroban, efegatran, or inogatran and

B) a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof for preventing or reducing a side effect linked to the use of one or more compound listed in A and/or for potentialising the therapeutic effect of one or more of said active agents.

The invention relates thus to a method of treatment where a patient is administrated:

A) one or more of the following compounds such as, heparin and heparin like compounds, synthetic heparins, synthetic heparin like compounds, low molecular heparins, ultra low molecular heparins such as oligosaccharides and pentasacharides such as fondaparinux sodium (Arixtra®), Idraparinux sodium, experimental ultra low molecular heparins, directs and indirects anti-Xa agents such as DX 9065a, anti factor IX agents, anti factor VII agents, directs and indirects anti coagulation factor agents, anti-IIa agents such as argatroban, ximelagatran (Exanta®), melagatran, lepirudin, recombinant hirudin and hirulog as and others direct thrombin inhibitors as hirudin, bivalirudin (such as Angiomax®), argatroban, efegatran, or inogatran and

B) a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof for preventing or reducing at least a side effect linked to the use of one or more compound listed in A and/or for potentialising the therapeutic effect of one or more of said active agents.

Preferably such method of treatment is applied to a patient in need, i.e. a patient suffering at least of a side effect linked to the use of one or more compound listed in A.

Preferably the side effect is bleeding or haemorrhage.

Advantageously, such method of treatment comprises the step of one or more test, preferably haemostasis tests. Such tests such as coagulation tests, aggregation tests, clotting tests, fibrinolysis tests, platelets tests, coagulation factors tests (such as anti-Xa & anti-IIa), bleeding tests, Azure A tests, allergy tests, immunologic tests, biologic balance tests, etc can be performed before, during or after compounds of list A administrations so as to determine the necessity or the need of administration(s) preventively (before), synergistically (during) or as antidote (after) of a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof.

In one embodiment such tests, as haemostasis tests, can help to determine the administrations paths of compounds of list A and/or list B and/or combinations thereof.

According to the invention, such haemostasis tests can be performed before, during and after the administration of compounds of list A as described above, compounds of list B and the combination thereof:

Test before A, Test before B, Test after A, Test after B, Test before and/or after the combinations of A and B.

According to the invention, such haemostasis tests can be performed before antidote administration and/or following antidote administration so as to determine the antidote dosage or successive necessary administrations and/or different necessary paths.

According to the invention, such successive necessary administrations of betaines compounds and/or different necessary paths, depending of the therapeutically purpose, can be carried out using different administrations modes of betaines compounds, said modes using different formulations and/or different concentrations and/or delivery rate and/or delivery speed and/or delivery devices and/or the combinations thereof.

The invention also relates to a method of treatment where a patient is administrated as effective antidote agent a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof for preventing, reducing or lessening a side effect linked to the use of one or more of the following compounds such as, heparin and heparin like compounds, synthetic heparins, synthetic heparin like compounds, low molecular heparins, ultra low molecular heparins such as oligosaccharides and pentasacharides such as fondaparinux sodium (Arixtra®), Idraparinux sodium, experimental ultra low molecular heparins, directs and indirects anti-Xa agents such as DX 9065a, anti factor IX agents, anti factor VII agents, directs and indirects anti coagulation factor agents, anti-IIa agents such as argatroban, ximelagatran (Exanta®), melagatran, lepirudin recombinant hirudin and hirulog as and others direct thrombin inhibitors as hirudin, bivalirudin (such as Angiomax®), argatroban, efegatran, or inogatran and compounds structurally similar to the preceding compounds.

In one embodiment, the invention relates to the process of administration of a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof before the administration of a second compound selected from one or more compounds such as, heparin and heparin like compounds, synthetic heparins, synthetic heparin like compounds, low molecular heparins, ultra low molecular heparins such as oligosaccharides and pentasacharides such as fondaparinux sodium (Arixtra®), Idraparinux sodium, experimental ultra low molecular heparins, directs and indirects anti-Xa agents such as DX 9065a, anti factor IX agents, anti factor VII agents, directs and indirects anti coagulation factor agents, anti-IIa agents such as argatroban, ximelagatran (Exanta®), melagatran, lepirudin recombinant hirudin and hirulog as and others direct thrombin inhibitors as hirudin, bivalirudin (such as Angiomax®), argatroban, efegatran, or inogatran and compounds structurally similar to the preceding compounds as to prevent at least one hemorrhagic side effect and/or for potentialising at least one therapeutically effect of one or more compounds selected from the second compounds.

In one embodiment, the invention relates to a composition or a pharmaceutical combination such as a kit comprising a therapeutic effective amount of a therapeutically active agent selected from:

A) one or more of the following compounds such as, heparin and heparin like compounds, synthetic heparins, synthetic heparin like compounds, low molecular heparins, ultra low molecular heparins such as oligosaccharides and pentasacharides such as fondaparinux sodium (Arixtra®), Idraparinux sodium, experimental ultra low molecular heparins, directs and indirects anti-Xa agents such as DX 9065a, anti factor IX agents, anti factor VII agents, directs and indirects anti coagulation factor agents, anti-IIa agents such as argatroban, ximelagatran (Exanta®), melagatran, lepirudin recombinant hirudin and hirulog as and others direct thrombin inhibitors as hirudin, bivalirudin (such as Angiomax®), argatroban, efegatran, or inogatran and

B) a therapeutic effective amount of a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof.

In one embodiment such a kit will be suitable for different administrations paths.

In one embodiment the methods of treatment of the invention relate to prophylaxis of deep-vein thrombosis, which may lead to pulmonary embolism:
For medical patients who are at risk for thromboembolic complications due to severely restricted mobility during acute illness;
For patients undergoing abdominal surgery who are at risk for thromboembolic complications;
For patients undergoing hip replacement surgery, during and following hospitalization;
For patients undergoing knee replacement surgery;
Prophylaxis of ischemic complications of unstable angina and non-Q-wave myocardial infarction, when concurrently administered with aspirin;
Inpatient treatment of acute deep-vein thrombosis with or without pulmonary embolism, when administered in conjunction with warfarin sodium and/or oral anticoagulant as thrombin inhibitors and others;
Outpatient treatment of acute deep-vein thrombosis without pulmonary embolism when administered in conjunction with warfarin sodium and/or oral anticoagulant as thrombin inhibitors and others.

Heparin is the most widely used intravenous (IV) anticoagulant and one of the most widely prescribed drugs in the World; for example more than 1 trillion units are administered each year to approximately 12 millions patients in US only, and dozen millions Worldwide. Indications for its use keep increasing. Unfortunately, the increased use of heparin has been accompanied by an increased occurrence of heparin induced thrombocytopenia (HIT). And in such patients alternative anticoagulants such as hirudin, lepirudin and recombinant hirudins are at increase use. The present invention is to provide a method of treatment of haemodialysis patients, anticoagulated with hirudin, lepirudin and recombinant hirudins where compounds of general formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof are used as bleeding antidote or anticoagulant reversing and/or antagonizing agents. The present invention claims betaines activities in inhibiting, reversing and/or antagonizing both direct and indirect thrombin inhibitors.

In one embodiment, the betaines can be used inside and/or to coat the membranes used in devices such as anticoagulant removal devices used in hemofiltration. Such membranes can be coated or can contain at least a betaine or compounds of general formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof. In one embodiment the betaine on the membrane can be used to scavenge others compounds from blood.

In one embodiment betaine due to its antiaggregant properties can be used to treat HUS (Hemolytic Uremic Syndrome) and/or thrombotic thrombocytopenic purpura (TTP).

In one embodiment betaine due to its antiaggregant properties can be used to treat heparin induced thrombocytopenia (HIT).

In one embodiment betaine (and the pharmaceutical compositions of the invention) due to its anti-haemorrhagic properties can be used to treat factor IX deficiencies such as Haemophilia and to treat Haemophilia bleeding. Betaines can also be combined to compounds used in the treatment of factor IX deficiencies to improve their pharmacological activities.

The invention relates also to a ready-to-use solution, whose administration does not require a reconstitution.

Any physiologically acceptable salt of the betaine may be used for preparing the solution of the invention. Examples of suitable salts may be, for instance, the salts with mineral inorganic acids such as hydrochloric, hydrobromic, sulphuric, phosphoric, nitric and the like, and the salts with certain organic acids such as acetic, succinic, tartaric, ascorbic, citric, glutammic, benzoic, methanesulfonic, ethanesulfonic and the like. The salt with hydrochloric acid is a particularly preferred salt. Most preferably, Glycine betaine used for the preparation of the solution is anhydrous glycine betaine or partly or completely hydrated glycine betaine, the glycine betaine being not in the form of a salt thereof, such as chloride, hydrochloride, etc. The glycine betaine is for example a synthetic betaine, but preferably a betaine extracted from a plant, such as beets, sugar beets, and purified up to a grade of more than 99.5%. While not being very clear, it seems that purified natural betaine has a better efficiency than synthetic glycine betaine.

Any solvent which is physiologically acceptable and which is able to dissolve the betaine salt may be used. The solution of the invention may also contain one or more additional components such as a co-solubilizing agent (which may be the same as a solvent), a tonicity adjustment agent and a preservative. Examples of solvents, co-solubilizing agents, tonicity adjustment agents and preservatives which can be used for the preparation of the betaine solutions of the invention are hereunder reported.

Suitable solvents and co-solubilizing agents may be, for instance, water; physiological saline; aliphatic amides, e.g. N,N-dimethylacetamide, N-hydroxy-2-ethyl-lactamide and the like; alcohols, e.g. ethanol, benzyl alcohol and the like; glycols and polyalcohols, e.g. propyleneglycol, glycerin and the like; esters of polyalcohols, e.g. diacetine, triacetine and the like; polyglycols and polyethers, e.g. polyethyleneglycol 400. propyleneglycol methylethers and the like; dioxolanes, e.g. isopropylidenglycerin and the like; dimethylisosorbide; pyrrolidone derivatives, e.g. 2-pyrrolidone, N-methyl-2-pyrrolidone, polyvinylpyrrolidone (co-solubilizing agent only) and the like; polyoxyethylenated fatty alcohols, e.g. Brij.sup.R and the like; esters of polyoxyethylenated fatty acids, e.g. Cremophor®, Myrj® and the like; polysorbates, e.g. Tweens®; polyoxyethylene derivatives of polypropyleneglycols, e.g. Pluronics®.

A particularly preferred co-solubilizing agent is polyvinylpyrrolidone.

Suitable tonicity adjustment agents may be, for instance, physiologically acceptable inorganic chlorides, e.g. sodium chloride, dextrose, lactose, mannitol and the like.

Preservatives suitable for physiological administration may be, for instance, esters of para-hydroxybenzoic acid (e.g., methyl, ethyl, propyl and butyl esters, or mixtures of them), chlorocresol and the like.

The above mentioned solvents and co-solubilizing agents, tonicity adjustment agents and preservatives can be used alone or as a mixture of two or more of them.

Examples of preferred solvents are water, ethanol, polyethyleneglycol and dimethylacetamide as well as mixtures in various proportions of these solvents. Water is a particularly preferred solvent.

To adjust the pH within the range of from 6 to about 8 a physiologically acceptable acid or base may be added as desired. The acid may be any physiologically acceptable base, e.g., a salt of an inorganic mineral acid such as hydrochloric, hydrobromic, sulfuric, phosphoric, nitric and the like, or an organic acid such as acetic, succinic, tartaric, ascorbic, citric, glutammic, benzoic, methanesulphonic, ethanesulfonic and the like, or also a physiologically acceptable buffer solution, e.g., a chloride buffer, an acetate buffer, a phosphate buffer and the like.

For obtaining pH values from about 6 to 8 the addition of a physiologically acceptable alkalinizing agent, such as sodium hydroxide, a mono, di- or triethanolamine or the like, or preferably, a buffer solution such as a phosphate buffer, a TRIS buffer or the like is required.

The preferred range of pH for the ready-to-use solution of the invention is from 6.5 to 7.5, in particular from about 7.

In the solutions of the invention the concentration of the betaine may vary within broad ranges, preferably from 0.1 mg/ml to 500 mg/ml, in particular from 1 mg/ml to 50 mg/ml, most preferably from 5 mg/ml to 20 mg/ml. Possible concentrations are 1 mg/ml, 5 mg/ml, 10 mg/ml, 10 mg/ml, 20 mg/ml, 40 mg/ml, 70 mg/ml, 85 mg/ml, 150 mg/ml, 200 mg/ml, 250 mg/ml, 300 mg/ml, 350 mg/ml 400 mg/ml, 450 mg/ml, 500 mg/ml etc. Higher concentrations are even possible but not preferred for human.

Suitable packaging for the betaine solutions may be all approved containers intended for parenteral use, such as plastic and glass containers, ready-to-use syringes and the like. Preferably the container is a sealed glass container, e.g. a vial or an ampoule. The vial can be provided with a sealing member or closure suitable to be pierced by the needle of the syringe.

According to a particularly preferred feature of the invention, there is provided a sterile, pyrogen-free, betaine solution which consists essentially of a physiologically acceptable betaine or salt thereof dissolved in a physiologically acceptable solvent thereof, which has a pH of from 5 to 8 and which is enriched in magnesium, sodium or potassium by addition of a physiologically acceptable salt.

In the above indicated preferred feature of the invention the physiologically acceptable salt of betaine may be, e.g. the salt with a mineral inorganic acid such as hydrochloric, hydrobromic, sulfuric, phosphoric, nitric and the like, or the salt with an organic acid such as acetic, succinic, tartaric, ascorbic, citric, glutamic, benzoic, methanesulfonic, ethanesulfonic and the like. The hydrochloride salt is a particularly preferred salt.

For the solution here above indicated as a preferred feature of the invention suitable solvents, co-solubilizing agents, tonicity adjustment agents and preservatives may be the same as those previously recited in this specification. Water is a particularly preferred solvent.

Also, the physiologically acceptable base which may be added to adjust the pH to from 5 to about 8, if desired, and the alkanilizing agent which may be added to adjust the pH, if desired, to a value from about 5.5 to 8.5 may be one of those previously specified.

Though the concentration of betaine in the above preferred feature may vary within the broad range from 0.1 mg/ml to 1000 mg/ml, preferred concentrations are from 2 mg/ml to 350 mg/ml, most preferably from 2 mg/ml to 250 mg/ml: examples of especially preferred concentrations of betaine are 1 mg/ml, 5 mg/ml, 10 mg/ml, 15 mg/ml, 25 mg/ml, 40 mg/ml, 70 mg/ml, 85 mg/ml, 150 mg/ml, 200 mg/ml, 250 mg/ml, 300 mg/ml, 350 mg/ml 400 mg/ml, 450 mg/ml, 500 mg/ml, 750 mg/ml, 1000 mg/ml.

The invention also provides a process for producing a sterile, pyrogen-free betaine solution with a pH of from 6 to 8. which process comprises dissolving a physiologically acceptable betaine or salt of the betaine, in a physiologically acceptable solvent thereof; optionally adding a physiologically acceptable base or buffer to adjust the pH within the said range as desired; and passing the resulting solution through a sterilising filter or to a filter followed by a sterilizing step.

One or more additional components such as co-solubilizing agents, tonicity adjustment agents and preservatives, for instance of the kind previously specified, may be added to the solution prior to passing the solution through the sterilising filter or the filtration step.

With the solutions of the invention it is possible to obtain compositions having a very high concentration of the betaine active substance even at 50 mg/ml and more.

The solutions of the invention are characterized by a good stability. Solutions in various solvents and with different PHs and concentrations have been found to be stable for long periods at temperatures accepted for the storage of pharmaceutical preparations.

Owing to the well known anti-thrombotic activity of the betaine active drug substance, the pharmaceutical compositions of the invention are useful as antidote against side action of heparin (or heparin like side effect) side effects in both human and animal hosts.

The injectable solutions of the invention are administered by intravenous injection or by slow infusion according to a variety of possible dose schedules. Suitable dose schedule for betaine may be, for example, of 10 to 300 mg of active drug substance per m² of body surface given as a single infusion and/or in repeated daily administrations, as long as required.

In one embodiment, the injectable solutions of the invention can be administrated preventively before further anticoagulant administration.

In one embodiment, the injectable solutions of the invention can be in the form where the betaines are spheronized and/or micro-coated as to augment their release time and/or to allow higher betaines concentrations such as 1000 mg/ml and more.

The compositions of the invention can further comprises at least one physiologically acceptable salt additive selected from the group consisting of sodium chloride, sodium hydroxide, sodium sulfate, magnesium chloride, magnesium hydroxide, magnesium sulfate, potassium chloride, potassium hydroxide, potassium sulfate and mixtures thereof, whereby the weight ratio glycine betaine/said at least one physiologically acceptable salt additive physiologically acceptable salts is advantageously greater than 3, preferably greater than 5, more preferably greater than 10, more specifically greater than 20.

The compositions of the invention can be possibly sealed in a container which has a layer in contact with the composition which is substantially free of Si atoms and/or of N atoms.

The compositions of the invention can be contained in a sealed container with a free volume corresponding advantageously to less than 20%, preferably to less than 10%, more preferably to less than 5% of the volume of solution.

The compositions of the invention can be submitted to a filtration with a filter with opening with a diameter lower than 0.5 μm, preferably lower than 0.3%, more preferably lower than 0.1 μm, more specifically lower than 0.01μ.

The compositions of the invention can have an osmolality at 20° from 150 to 1000 mOsm/kg, advantageously from 200 to 750 mOsm/kg, preferably from 225 to 500 mOsm/kg, more preferably from 250 to 350 mOsm/kg, more specifically from 270 to 330 mOsm/kg.

The compositions of the invention can have a viscosity comprised between 0.1 and 100 m Pa·s at 20° C. advantageously comprised between 0.5 and 50 m Pa·s, preferably comprised between 0.5 and 25 m Pa·s more preferably comprised between 0.75 and 10 m Pa·s more specifically comprised between 1 and 5 m Pa·s.

The compositions of the invention can comprise water as physiologically acceptable solvent, said water containing less than 200 ppm salts, advantageously less than 150 ppm salts, preferably less than 100 ppm salts, more preferably less than 50 ppm salts, more specifically less than 25 ppm salts.

The injectable solutions of the invention can be also be administrated subcutaneously or by intramuscular way according to different paths of administration depending of the aimed therapeutically effect.

In one embodiment the invention describes a pharmaceutical unit dosage form of a composition containing at least a betaine in a form of a gel, said dosage form being selected from the group consisting of sachets, pouches, blisters and bags.

In one embodiment, the solutions of the invention after to have been tested with the methods of the invention (Azure A) can possibly further be submitted to one or more drying processes. Such additional drying processes allowing the recovery of one or more betaine in a solid phase (i.e. the water or liquid removed partially or completely), such betaine in a solid phase being further used in the fabrication of a medicament characterized by:

Pharmaceutical unit dosage form of a composition containing at least a betaine, said dosage form being selected from the group consisting of sachets, pouches, blisters and bags, wherein the pharmaceutical unit dosage form is provided with moisture barrier property defined by an increase of weight of the composition of less than 1% after storage of the unit dosage form in sealed condition in an environment with a temperature of 38° C. and a relative humidity of 90% during 30 days.

Such individual sachet being possibly further submitted to an encrypting against counterfeiting and/or a notch to facilitate the tearing and/or the opening.

As MVTR stands for “Moisture Vapor Transmission Rate”, a measure of the passage of gaseous H₂O through a barrier, the pharmaceutical oral unitary dose of betaine in a sealed dosage form from the group consisting of sachets, bags, blisters and pouches in which the dosage form is at least partly flexible, water impermeable and characterized by a protective barrier by a MVTR value inferior to 0.1 g/m² at 38° C. and 90% relative humidity during 24 hours.

In one embodiment, the sizes of the betaines particles can be selected so as to absorb minimally the water (for instance from micronized particles to an optimal size particles allowing a minimal water intake). Optionally the particles (or the dosage form such as a sugar-coated pill could be further enveloped by a surfactant having good moisture barrier) can be sugar-coated and optionally such particles can be trapped in a gel or a polymer before being packaged in a selected MVTR container or pharmaceutical unit dosage form.

For example the coating or primary packaging material could be a laminate which is made up of 12 μm PET, 25 μm Alufoil and a 50 μm PE inner heat-seal layer. Further high quality and clarity of surface decoration might be realized by gravure reverse printing process.

The complete barrier requirement for this highly hygroscopic product (betaine after being dried, i.e. its liquid content partially or completely removed) could be provided by a laminate of PET, PE and Alufoil. The single 250 to 5000 mg doses are easy to tear open and safe for mouth contact. In this dosage form betaine can be taken directly by mouth without the need to dissolve in water. Some flavoring agents might be added to mask betaines taste by the way augmenting patients' compliance. Geometrical forms which augment the facility of use can be privileged.

In one embodiment a stick format of the sachet will be preferred as it uses a minimum amount of material in relation to the volume of its contents and further by reducing the bag surface it allows also to reduce the MVTR.

The dosage forms can be optimized according to selected combinations of MVTR, betaine doses, tensile strengths, sizes, forms, coefficients of friction. The initial rate of moisture of the betaines can also be selected and/or controlled so as to lower the other parameters (MVTR, etc) thus augmenting the compliance of the dosage form. Betaine monohydrate and/or Betaine anhydrous and/or theirs mixtures solutions after being submitted to the processes of the invention can be dried and sachets as unit oral dosage forms of betaine(s) can be realized using as primary packaging material multilayer Alufoil material. The realized sachets will be weighted just after their manufacture and 1, 3, 6 and 12 months later, so as to determine the possible water intake of the betaine(s) inside the sealed dosage forms. The results could show that the weight variation is in accordance and in the limits allowed by the International Pharmacopoeia and come up to the FDA and/or BGA directives and the recommended and approved instructions given by EF for this kind (sachets/blister/pouches) of pharmaceutical dosage form. Due to the high hygroscopic properties of betaines, in one embodiment betaine monohydrate can be preferred. In effect the water intake of betaine monohydrate can be in a preliminary step controlled so as

In one embodiment the process (Azure A determination) of the invention can optionally be omitted when Betaine monohydrate and/or Betaine anhydrous and/or theirs mixtures are used “as is”, i.e. as provided in pharmaceutical grade (suitable for oral use in nutritional products) by the manufacturers after the sugar beet molasses separation processes or the chemical or biological synthesis. These betaines edible nutritional products can be the packaged in such unit oral dosage form sachets having such moisture and/or oxygen and/or light barriers and/or tensile strength and/or coefficient of friction.

The coefficients of friction outside/outside and/or inside/inside will have to be carefully chosen so as to allow the maximum of the compound to be delivered at the administration. When absorbing moisture the betaine can start a process of higher size crystallization which can adhere inside the sachet making a part of the betaine unavailable upon administration. In the other side such medicament being destined to a daily utilisation during years, it is necessary to carefully choice the physical properties of the primary packaging material so as to have at the same time a good moisture barrier while having an easy opening, i.e. a “friendly” tensile strength allowing for instance elderly people to take easily their daily or twice daily medication. The primary packaging material must be easy to tear while possessing good moisture and/or oxygen barriers, such barriers preventing betaines deliquescence. Moisture sorption could lead to betaine particles agglomeration which can then adhere inside the sachet leading to a partial delivery of the drug after tearing. When carefully chosen, selected and combined these parameters will allow compliance with the International Pharmacopoeia and Pharmaceutical Industry standards as they (parameters) will allow a better compliance of the end user, i.e. the patient. All the combinations do not work and only a careful selection of specific materials with particular parameters can provide this double compliance of sachet oral unit dosage forms of betaines.

Thus it is claimed here the combinations of the above physical characteristics of betaines forms (salts, sizes, coatings, polymers, etc) and the physical characteristics of the packaging materials (MVTR, tensile strength, coefficient of friction, tear strength, etc) which (the combinations) allow to augment the compliance of the pharmaceutical dosage form while retaining and respecting correct (according to international regulations and directives) conservation properties.

Further the compositions of the inventions can be used and are claimed to be useful to treat, prevent or alleviate the clinical signs of the following pathologies:

Lupus anticoagulant, miscarriage, pregnancy, antiphospholipid syndrome, thrombotic and/or obstetric complications, specially miscarriages and/or repeated fetal deaths, pregnancy, intra- and postpartum, hemorrhoids anticardiolipin antibodies, primary and/or secondary haemostatic disorders, prevention in travel induced thrombosis such as air travel deep venous thrombosis, chronic venous insufficiency CVI grade I or II Widmer classification, heavy legs, portal hemorrhage, portal hypertension, pulmonary hypertension, bleeding of oesophageal varices, sepsis and severe sepsis, coagulopathy, namely disseminated intravascular coagulation (DIC), complications in sepsis, uncontrolled cascade of coagulation, fibrinolysis, inflammation, Nash & liver diseases, homocystinuria & homocysteinemia, sepsis, septic shocks, bleeding, hypertension, pulmonary hypertension, intermittent claudication, portal hypertension, hypertension, vascular hypertension, ocular hypertension, gangrene, diabetes, cardiovascular diseases, heart diseases, angina pectoris, atrial fibrillation, cerebrovascular diseases, peripheral arterial diseases, inflammation diseases, kidney diseases, cancer diseases, sexual dysfunction and metabolic syndrome.

Portal hypertension is an increase in the blood pressure in the portal vein, which carries the blood from the bowel and spleen to the liver. The pressure in the portal vein may rise because there is a blockage, such as a blood clot, or because the resistance in the liver is increased because of scarring, or cirrhosis. As a result, the pressure in the portal vein rises—this is known as portal hypertension.

Betaine according to the invention is used both for the prevention of bleeding and also in those people who have bled. It may be used in the prevention of re-bleeding.

Sepsis can be defined as a spectrum of clinical conditions caused by the immune response of a host to infection or trauma and characterized by systemic inflammation and coagulation (Mesters, 1996a; Wheeler, 1999). It ranges from a systemic inflammatory response to organ dysfunction to multiple organ failure, and ultimately death for many patients.

In simplified terms, sepsis can be conceptualized as a dysfunction of the opposing mechanisms that normally maintain homeostasis. On one side are increased inflammation and coagulation, which are driven by proinflammatory mediators, endothelial injury, tissue factor expression, and thrombin production (Vervloet, 1998; Hesselvik, 1991; Kidokoro, 1996; Levi, 1997; Carvalho, 1994). On the opposite side is suppressed fibrinolysis, which normally counters procoagulant forces (Vervloet, 1998; Kidokoro, 1996).

The betaines of the invention are claimed to be particularly suitable for the treatment of the various sepsis pathologies.

Chronic venous disease (CVD) is defined as abnormal functioning of the venous system caused by venous valvular incompetence, which may affect the superficial or deep venous system or both.

The betaines of the invention are also claimed to be particularly suitable to reduce the accumulation of blood in the veins and edemas and improve venous return. They can alleviate the symptoms of well-established varicose veins or varicosities and could give relief from the symptoms of venous disease, prevent its evolution, and are less constricting than retention.

By reducing the distension of the veins, they facilitate their emptying and reduce venous stasis.

In terms of microcirculation, they regulate the permeability of the capillaries by increasing their resistance. As the blood pressure increases, the small vessels allow liquid to escape, which creates edema. One of the objectives of betaines of the invention is to limit this leakage.

Indeed the betaines of the invention, optionally micronized, prevent the evolution of the disease by preventing the inflammation of the endothelium at the level of both microcirculation and the valves.

In one embodiment the betaines, preferably in the oral form, can be administrated before and/or during and/or after travel to prevent the pathologies linked to long or short hauls such as thromboembolism, DVT, heavy legs, cramps, oedemas, limb trauma, pulmonary embolus, oral contraceptives or hormonal replacement risks, previous DVT, history of malignancy, recent surgery, history of inflammatory bowel disease, familial/hereditary risk factors, immobilization, obesity, smoking, cramped seating, recent trauma from an accident or surgery, decreased oxygen, etc.

In one embodiment the betaines, preferably in the oral form as a sachet, can be presented or manufactured as a kit suitable for instance to an administration the day before, the day during and the day after the travel or the journey. The dosage for the return journey can of course also be previewed.

In one embodiment, the betaines can be mixed to meals, food bars, cookies, drinks, confectioneries, sweets, sweeteners, biscuits and in any form or association with comestible ingredients suitable for oral ingestion. Such nutritional forms of betaines suitable for oral and enterally nutrition and having the pharmacological properties of betaines claimed in the present specification.

The following examples illustrate but do not limit in any way the invention.

### EXAMPLES

### Example 1

The compositions having been prepared:
from synthetic glycine betaine (purity of more than 99.9% by weight) or from purified natural betaine (purity of more than 99.7% by weight);
purified water for injection
sodium chloride
sodium hydroxide

The amount of sodium hydroxide added was for adapting the pH of the solution.

The pH of the solution was adjusted. Further de-aerated water for injections was added to bring the solution to its final volume or concentration.

The solution was filtered through a 0.22 μm microporous membrane under nitrogen pressure. Volumes of 10, 25 and 50 ml of the solution were distributed into type I-colourless glass vials. The vials were then closed with chlorobutyl Teflon-faced rubber stoppers and sealed with aluminium caps.

The following table gives the content of compounds per ml of solutions.

| | | | sodium | weight ratio betaine/ |
|---|---|---|---|---|
| | betaine | sodium | hydroxide for | sodium based |
| example | mg/ml | chloride | a pH of | compounds |
| 1 | 5 (s) | 1 | 7 | about 5 |
| 2 | 10 (s) | 2 | 7 | about 5 |
| 3 | 20 (s) | 4 | 7 | about 5 |
| 4 | 50 (s) | 10 | 7 | about 5 |
| 5 | 10 (n) | 2 | 7 | about 5 |
| 6 | 20 (n) | 4 | 7 | about 5 |
| 7 | 50 (n) | 10 | 7 | about 5 |
| 8 | 5 (s) | 1 | 6.5 | about 5 |
| 9 | 10 (s) | 2 | 7.5 | about 5 |
| 10 | 20 (s) | 4 | 6 | about 5 |
| 11 | 50 (s) | 10 | 6.5 | about 5 |
| 12 | 10 (n) | 2 | 6.5 | about 5 |
| 13 | 20 (n) | 4 | 8 | about 5 |
| 14 | 50 (n) | 10 | 6.5 | about 5 |
| 15 | 5 (s) | 0.5 | 7 | about 10 |
| 16 | 10 (s) | 1 | 7 | about 10 |
| 17 | 20 (s) | 2 | 7 | about 10 |
| 18 | 50 (s) | 5 | 7 | about 10 |
| 19 | 10 (n) | 1 | 7 | about 10 |
| 20 | 20 (n) | 2 | 7 | about 10 |
| 21 | 50 (n) | 5 | 7 | about 10 |

s: synthetic
n: natural

The sealed vials were submitted to a sterilization step, such as heating step at 121° C. during 5 to 60 minutes, irradiation (Gamma irradiation), etc. Just after the sterilisation step, the vials were submitted to a shaking, for example as long as the temperature of the liquid is above 60° C.

When no shaking was carried out after the sterilisation step at 121° C., a drop of osmolality was observed. The drop of osmolality was lower for solution with a high weight ratio betaine/(NaCl+NaOH), such as ratio above about 7 or for solution with a betaine concentration of more than about 25 mg/ml, such as 50 mg/ml, 100 mg/ml or more. Such a drop was also low for betaine solutions with a PH lower than 6.0. When drop of osmolarity is low no shaking is necessary.

The examples were repeated except that a mixture of NaCl (50% by weight) and KCl (50% by weight) was used instead of NaCl.

### Example 2

Realisation of Iso-Osmotic Pure Betaine Solutions at 40 mg/ml

Material:

Betaine monohydrate BETAFIN AP-DANISCO-Batch N° 50000451-06.03.2003
Autoclave Sanoclav type KL-12-3. serie A-2452-02.
Automatic Osmometer Knauer
Classed I Brown glass vials, countenance 5 ml (vials Macherey-Nagel, art. 702.15.36)
Magnetic agitator
Peristaltic pump (Baxa Fluid Tranfer Tube Set Ref 11. Lot 132562005)
Water for injection (Baxter Viaflo 500 ml, Lot 02104E1N)
2 Media-Kap filters from 2 different lots (75957B; 75933A)
   Solutions Preparation:

In a sterile and apyrogen 500 ml gauge, 20.0657 g monohydrate betaine are supplemented with pyrogen free water as to obtain final volume of 500 ml of solution which is homogenized under magnetic agitation.

The vials and the butyl caps are washed and sterilized, 133° C. vapour cycle (all the Thermalog® wired). The Peristaltic pump is mounted with a transfer tube set cut at its extremities with sterile scissors, one of the extremities been dived in the gauge the other been mounted in a serial manner with the two Media-Kap filters from 2 different lots.

Each vial is filled precisely with 4.4 ml of the betaine solution.

Characterization of the Initial Solution:

Colourless and limpid solution
Osmolality (Knauer Osmometer Automatic): 324±1 mOsm/kg (n=3)
pH: 7.51
   Autoclave Process—Solution A

Each vial been filled precisely with 4.4 ml of the betaine solution before been sealed with the butyl cap and further sealed with metallic caps.

The vials are placed in the autoclave and submitted to a sterilization test at 121° C. during 30 minutes.

Characterization of the Obtained Autoclaved Solution:

Colourless and limpid solution Osmolality
(Knauer Osmometer Automatic): 329.3±2.5 mOsm/kg (n=3)
pH: 7.13
   Filtration Process—Solution B

Aseptic solution the obtained solution is submitted under laminar flux to a filtration with a 0.22 μm filter membrane and the filtrate is conditioned in sterile vials. Each vial been filled precisely with 4.4 ml of the betaine solution

### Example 3

Characterization of Binding Activity to Glycosaminoglycans of Different Solution Using the Azure A test.

Because of the drop of osmolality and pH after the autoclave process, it is interesting to know which of the solutions retains its physical property to bind to heparin, such ability been predictive of in vivo pharmacological efficacy of the claimed solutions.

Procedure:

Azure A method is a rapid and simple spectrometric method for determination of heparin concentration following the formation of soluble complex between heparin and azure A dye. The principle of the test rests in the spectrometric follow up at 632 nm wave length of an azure A/heparin complex. Azure A is a basic blue dye which when combined to heparin changes its colour to purple. Chemically, azure A is formed by 3 benzene nuclei and a terminal N⁺ which binds to heparin negative charges. The detection principle is based on the lower absorbance recorded following heparin bounding to azure A in the mixed solutions, the latter turning in deeper purple as heparin concentrations are higher. In the presence of a substance which binds to heparin, for instance protamine or here Betaine, azure A retains its blue colour and absorbance remains optimal. Betaine solutions tested in this setting showed different binding properties depending on theirs origins, for example synthetic or natural, and depending also on theirs manufacture processes for instance the sterilization processes used as autoclave or microfiltration. The differences obtained in the solutions absorbances can predict in vivo pharmacological efficiencies, and can allow best betaine solutions selection.

Solutions Preparations:

Azure A

Sigma Aldrich—CAS Number 531-53-3

Stock solution preparation at 4×10⁻⁵ mol L⁻.
1) 58.35 mg azure A are solubilized in 50 ml sterile injectable water
2) take 10 ml of this solution and dilute it in 90 ml sterile injectable water
3) take 10 ml of this second solution and dilute it in 90 ml sterile injectable water
4) the obtained solution is filtered on Watman paper & aliquots of 4 ml are realised
   Heparin

Heparin CHOAY stock solution at 5000 UI/ml as provided by the manufacturer (Sanofi Pharma—Heparin CHOAY) CAS Number: 9005-49-6

1 ml of this solution is diluted in 499 ml sterile injectable water to obtain 10 UI/ml (IU=International Unit) in final concentration, this solution will be used for the reference spectrometric follow up of heparin binding to Azure A.

Betaine

Betaine monohydrate—CAS Number 17146-86-0 is diluted in sterile injectable water as to obtain a 12.5 mg/ml solution in final concentration.

Betaine/Heparin Combination

0.8 ml of betaine solution is mixed with 1.2 ml heparin stock solution corresponding to the mix of 10 mg betaine to 6000 IU heparin (+ to 40 mg heparin). The obtained 2 ml of this combined solution are mixed under agitation during 2 minutes and then incubated during 10 minutes at 20° C. before being diluted in 998 ml injectable water as to obtain final combination solution.

Tests:

Azure A solution will serve to settle the 100% absorbance (blue colour) by spectrometry at 632 nm length wave.

Heparin+Azure A and Heparin+Betaine solutions absorbances are realized according to the following scheme:

Aliquot 400 μl solution to test+4 ml solution azure A

Results:

Solution A—Autoclaved at 130° C. during 40 minutes (overkill procedure)

Solution B—Filtration at 0.22 μm filter cut off

| | |
|---|---|
| Azure A + Water | 1.370 |
| Azure A + Heparin | 0.465 |
| Azure A + Betaine A + Heparin | 0.867 |
| Azure A + Betaine B + Heparin | 1.088 |

The autoclaved betaine (Betaine A) solution seems having lost a part of its ability to bind heparin comparatively to the filtered solution (Betaine B) as shown by its lower absorbance, meaning that less autoclaved betaine bound to heparin thus allowing the remaining free portion of glycosaminoglycans to bind to azure A modifying its absorbance. It is important to know exactly the physical property of each solution, since in clinical setting when the solutions safety attained it is necessary to have an efficient and reliable medicine in acute situations such as anticoagulants induced bleedings or haemorrhages.

Fractioned heparins were also assayed in the test:

| | |
|---|---|
| Azure A + Arixtra | 0.892 |
| Azure A + Betaine B + Arixtra | 1.185 |
| Azure A + Lovenox | 0.253 |
| Azure A + Betaine B + Lovenox | 0.248 |

As glycosaminoglycans incorporate also fractioned heparin such as enoxaparin sodium and fondaparinux sodium have been tested, with azure A test. From this test it appears that when using fractioned heparin instead of unfractionned heparin in the azure A test of the invention a spectrometric value of 0.9 at 632 nm wave length value, can be achieved although the betaine B was an effective antidote for said unfractionned heparin (Arixtra & Lovenox). The skilled man was unable to predict that a betaine fulfilling the 0.9 requirement of the test of the invention (with unfractionned heparin) was effective as antidote for fractioned heparin as Arixtra & Lovenox although the test with fractioned heparin give spectrometric values far below than the 0.9 obtained with unfractionned heparin. The results from the above table establish clearly that no predictability can be achieved when using a fractioned heparin, although said fractioned heparin might have good in vivo pharmacological efficiency.

Biological Activity

### Example 4

Evaluation of Biological Activity of a Synthetic Betaine Solution

| | LASER | | | AGGREGATION | | |
|---|---|---|---|---|---|---|
| | | Numb- | | Am- | | Bleeding |
| | Laser | er of | Duration | plitude | Velocity | Bleeding |
| | shoots | emboli | minutes | ohms | Ohm/min | (seconds) |
| Rat # 1 | 1 | 9 | 4 | 7 | 9 | 115 |
| Rat # 2 | 2 | 7 | 3 | 9 | 9 | 120 |
| Rat # 3 | 2 | 6 | 3 | 10 | 9 | 110 |
| Rat # 4 | 2 | 2 | 1 | 4 | 7 | 360 |
| Rat # 5 | 2 | 5 | 2 | 9 | 8 | 125 |
| Rat # 6 | 2 | 6 | 3 | 12 | 11 | 110 |
| Rat # 7 | 2 | 4 | 2 | 9 | 8 | 115 |
| Mean | 1.86 | 5.57 | 2.57 | 9.14 | 9.71 | 150.71 |

The tests clearly show that synthetic betaine has less pharmacological activity, namely antithrombotic activity than natural betaine on basis of previous experiments in this experimental thrombosis model. Natural betaine showed much better antiaggregant and antithrombotic activity and less bleeding tendency than these exhibits in the present test by the synthetic betaine.

### Example 5

Comparative Effect of Betaine Solutions

Animals

Twelve (12) male Wistar rats from Charles River—France, were acclimated for one week before the tests. They were then weighed prior to the test (weights ranged from 270 to 350) and divided in 2 groups of 6 animals.

Each group being subcutaneously administrated 1 hour before the tests Solution A=autoclaved or Solution B=micro-filtered of example 2 at the dose of 15 mg/kg.

Experimental Procedure.

Principle of Laser-Induced Thrombosis

(Seiffge D. et al., 1989; Weichter W. et al., 1983)

In this model, lesion of the vascular wall is induced by a laser beam. This beam causes a limited lesion of the vascular endothelium (only 1 to 2 cells are destroyed).

This laying base of the sub-endothelium, which is a thrombogenetic surface, triggers the adherence of platelets via glycoproteins. This adherence of platelets is followed by their activation; they form pseudopods and secrete the content of their granules. This activation results in the appearance of glycoprotein binding sites which are necessary for the aggregation of the platelets between them and for platelet adhesion to the thrombogenic surfaces. This lesion is induced in the mesenteric microcirculation of the rat. It is immediately followed by the formation of a thrombus (in a few seconds). This thrombus, which rapidly enlarges under the influence of the blood flow, embolises before being formed again. In this model, an antithrombotic compound shows more laser shoots and less emboli (thrombus)

Induced bleeding time

(E. Dejana. Bleeding time in rats. Thrombosis Research. 1982)

Blood samples are made before the test. The tail of anaesthetised rat is dipped for 5 minutes in a water bath at 37° C. so as to provoke a dilatation of the peripheral vessels; then the tail is removed and cut at the end (5-7 mm from the tip), the chronometer being started. The induced bleeding time is defined as being the time period comprised between the cutting of end tail and the end of the haemorrhage or bleeding. The end of haemorrhage is defined as the time where the last drop of blood is removed from the tail and no other drop is seen during 180 seconds. The substances were subcutaneously administrated 60 minutes prior to the tail cut.

Antithrombotic and Bleeding Activities

| | Number of | | Number of | | Duration of | | Bleeding | |
|---|---|---|---|---|---|---|---|---|
| | laser shoots | | emboli | | embolisation | | (seconds) | |
| | A | B | A | B | A | B | A | B |
| Rat 1 | 3 | 3 | 1 | 1 | 0 | 0 | 115 | 90 |
| Rat 2 | 2 | 4 | 2 | 1 | 1 | 0 | 105 | 100 |
| Rat 3 | 4 | 2 | 0 | 1 | 0 | 0 | 105 | 65 |
| Rat 4 | 2 | 4 | 1 | 0 | 0 | 0 | 105 | 105 |
| Rat 5 | 3 | 4 | 1 | 0 | 0 | 0 | 100 | 115 |
| Rat 6 | 3 | 3 | 1 | 1 | 0 | 0 | 95 | 110 |
| Mean | 2.83 | 3.333 | 1 | 0.67 | 0.17 | 0 | 104.17 | 97.5 |

A = Autoclaved solution A
B = Filtered solution B

Biological Balance

| | | |
|---|---|---|
| Aptt | Quick | Fibrinogen |

| | A | B | A | B | A | B |
|---|---|---|---|---|---|---|
| Rat 1 | 55.4 | 61.2 | 38.4 | 39.4 | 1.33 | 1.4 |
| Rat 2 | 49.3 | 53.4 | 37.2 | 37.5 | 1.40 | 1.15 |
| Rat 3 | 60.5 | 69.1 | 40.3 | 44.7 | 1.96 | 1.53 |
| Rat 4 | 44.4 | 51.4 | 33.2 | 34.6 | 1.37 | 1.41 |
| Rat 5 | 57.3 | 66.2 | 39.6 | 41.8 | 1.61 | 1.44 |
| Rat 6 | 56.1 | 57.3 | 35.4 | 37.8 | 1.53 | 1.51 |
| Mean | 53.83 | 59.77 | 37.35 | 39.3 | 1.53 | 1.41 |

The autoclaved solution A, shows less pharmacological activity than the filtered solution B, confirming the results of the Azure A test of example 3.

### Example 6

Bleeding and Blood Loss Following Enoxaparin, Betaine Effect.

Background and Purpose

The aim of the study was to investigate if the delayed administration of Betaine solution (antidote type of administration) could decrease the bleeding time and reduce the blood loss volume induced by Enoxaparin treatment in rat.

Chemical products

Betaine Solution B
Sterile water, injectable solution (Aguettan, France)
Enoxaparin sodium—Lovenox®—Rhône Poulenc-Rorer (±100 IU/mg)
Anaesthetic, Nesdonal (Rhône Poulenc, France)
   Animals

Nineteen (19) male Wistar rats from Charles River—France, were acclimated for one week before the tests. They were then weighed prior to the test (weights ranged from 240 to 320. mean 275 g) and divided in 3 groups of 6 or 7 animals.
Experimental protocol (E. Dejana. Bleeding time in rats. Thrombosis. Res. 1982)

The different treatments are summarised in following table.

| | treatments | | |
|---|---|---|---|
| groups | T0 | T0 + 30 min | T0 + 60 min |
| Enoxaparin | Enoxaparin 5 mg/kg | Saline | bleeding test |
| (n = 6) | | | |
| Antidote I | Enoxaparin 5 mg/kg | Betaine 10 mg/kg | bleeding test |
| (n = 7) | | | |
| Antidote II | Enoxaparin 5 mg/kg | Betaine 15 mg/kg | bleeding test |
| (n = 6) | | | |

The experiment was performed according to the previously described methodology. The tails were sectioned at 6 to 10 mm from the extremity. After transection the proximal end of the tail was placed into a tube and blood was permitted to drip freely into a reservoir of 3.8% citrate solution (1 ml) till bleeding stop, then blood loss volumes were determined using a 1000 μL pipette. At the end of experiment, before euthanasia, blood was sampled by intracardiac puncture on Na-citrate (3.8%, 1:9) and centrifuged at 4000 tours/min during 20 min as to obtain Poor Platelet Plasma (PPP). Plasmas are kept at −20° C. against future biochemical assays.

Results

Bleeding time and Blood loss.

Saline control values (previous experiments): Bleeding time ˜110 seconds-Blood loss ˜250 μL

| | Enoxaparin | | | | | |
|---|---|---|---|---|---|---|
| | 5 mg/kg | | Enoxaparin 5 mg + | | Enoxaparin 5 mg + | |
| | Bleed- | | Betaine10 mg | | Betaine 15 mg | |
| | ing time | Volume | Bleeding | Volume | Bleeding | Volume |
| | (sec) | μL | time (sec) | μL | time (sec) | μL |
| Rat # 1 | 235 | 800 | 165 | 500 | 125 | 350 |
| Rat # 2 | 310 | 1100 | 140 | 450 | 130 | 340 |
| Rat # 3 | 300 | 900 | 170 | 600 | 145 | 440 |
| Rat # 4 | 410 | 1200 | 135 | 400 | 100 | 330 |
| Rat # 5 | 290 | 850 | 135 | 550 | 125 | 350 |
| Rat # 6 | 310 | 930 | 120 | 330 | 75 | 150 |
| Rat # 7 | — | — | 155 | 600 | — | — |
| Mean | 309.17 | 963.33 | 145.71* | 490* | 116.67** | 326.67** |
| S. Dev. | ±56.78 | ±154.49 | ±18.13 | ±102.63 | ±25.03 | ±95.22 |

*P < 0.001 vs. Enoxaparin
**P < 0.005 vs. Enoxaparin (Student)

Comments

Betaine injectable solution completely normalized bleeding time prolonged by enoxaparin administration. It also restored blood loss near to saline control values.

These results open the door to new indications for LMWH since when possessing an efficient antidote theirs uses could be extended to acute situations as cardiovascular bypass or others. Betaines utilisation as antidote, due to theirs efficiencies and safeties can open the door to low molecular heparins as to synthetic and natural oligosaccharides and pentasacharides, for extended utilisations i.e. the same utilisations as unfractionned heparins. A kit containing a LMWH with its antidote could be helpful in acute clinical situations.

### Example 7

Betaine Solution Effect on Fondaparinux Sodium Anti-Xa Activity.

Introduction

Arixtra® is a pentasacharide with a size of 1728 Daltons and a half life up to 200 minutes when administrated s.c to rats.

We expected that Betaine effect vs. Arixtra® can be measured through Anti-Xa assay. This could be clearly helpful in patients' anticoagulation monitoring, after Betaine a ministration. The aim of this series of experiments was to evaluate Betaine effect in situation which closely mimics clinical conditions where a patient been administrated Arixtra® is in need to have its anticoagulation reversed.

Experimental Protocol

Rats from Charles River—France, were housed and acclimated during 7 days. Prior to tests they were weighed (weights ranged from 300 to 350 g) and anaesthetised by intramuscular injection of Nesdonal (200 mg/kg). Arixtra® vial (fondaparinux sodium) at 2.5 mg/0.5 ml was diluted in 6.5 ml saline. The obtained solution was intravenously administrated to rats at 1 ml/kg (0.357 mg/ml/kg), via the penile vein at T0. Then through a medial longitudinal incision in the neck, jugular veins where carefully exposed and catheterised for blood samplings; the other sides of the catheters were closed with sampling syringes filled with 170 μL sodium citrate 3.8%. At T0+30 min, samples of 1.5 ml blood were taken and new sodium citrate syringes been placed on the catheter lines. Then the iso-osmotic betaine solution B of example 2, at 40 mg/ml was subcutaneously administrated (15 mg/kg) to rats. At T0+50′ and T0+70′ the 1.5 ml blood sampling operations were repeated, followed by rats' euthanasia.

Blood samples were centrifuged at 4000 tours/min during 20 min as to obtain Poor Platelet Plasma (PPP). Arixtra was considered to have Anti-Xa activity of ±850 IU/mg. Anti-Xa activities were determined using a chromogenic assay and Automatic Coagulation Laboratory ACL 200® (Instrumentation Laboratory, France).

| | Timing | | | | |
|---|---|---|---|---|---|
| | T0 | | T0 + 30′ | | |
| | Arixtra ® | T0 + 30′ | Betaine | T0 + 50′ | T0 + 70′ |
| Treatment | 0.357 mg/kg | Blood | 15 mg/kg | Blood | Blood |
| | IV | sample | sc | sample | sample |

Results

| | Anti-Xa U/ml | | |
|---|---|---|---|
| | Sample 30′ | Sample 50′ | Sample 70′ |
| Rat # 1 | 1.95 | 0.65 | 0.60 |
| Rat # 2 | 2.20 | 0.84 | 0.75 |
| Rat # 3 | 2.00 | 0.76 | 0.70 |
| Rat # 4 | 1.76 | 0.65 | 0.60 |
| Rat # 5 | 2.30 | 0.98 | 0.85 |
| Rat # 6 | 2.12 | 0.73 | 0.62 |
| Rat # 7 | 1.97 | 0.55 | 0.49 |
| Rat # 8 | 2.37 | 0.74 | 0.62 |
| Rat # 9 | 2.21 | 0.88 | 0.69 |
| Mean | 2.098 | 0.753 | 0.658 |
| S. Deviation | ±0.19 | ±0.13 | ±0.10 |

Comments

At T0+70′ the invention iso-osmotic solution of Betaine at 40 mg/ml reduced Arixtra's® Anti-Xa activity by 69%. This reduction occurred uniformly in all animals

This study was designed to avoid as much as possible bias and artefacts, each animal being its own control. Results show clearly that Betaine have reversing effects on Arixtra® anticoagulation. Increasing Betaine doses are expected to provide higher or complete reversals since there is a dose effect with betaine utilisation. Betaine pharmacokinetics follow up in animals and human had shown a Betaine activity and half life during long periods of time, this been really beneficial in case of in vivo reversal of compounds with a long half life as for example Fondaparinux or Idraparinux sodium.

### Example 8

Betaine Solution Effect on ULMWH Anti-Xa Activity.

ULMWH is an experimental oligosaccharide with a size of less than 2000 Daltons and a half life up to 200 minutes when administrated s.c to rats at 5 mg/kg.

Previous experiments showed that Betaine effect vs. ULMWH can be measured through Anti-Xa assay. This could be clearly helpful in patients' anticoagulation monitoring, after Betaine administration. The aim of this series of experiments was to evaluate Betaine effect in situation which closely mimics clinical conditions where a patient been administrated ULMWH is in need to have its anticoagulation reversed.

Experimental Protocol

Eleven rats from Charles River—France, were housed and acclimated during 7 days. Prior to tests they were weighed (weights ranged from 300 to 350 g) and anaesthetised by intra-muscular injection of Nesdonal (200 mg/kg). ULMWH (5 mg/ml/kg) was intravenously administrated via the penis vein at TO. Then through a medial longitudinal incision in the neck, jugular veins where carefully exposed and catheterised for blood samplings; the other sides of the catheters were closed with sampling syringes filled with 170 μL sodium citrate 3.8%. At T0+30 min, samples of 1.5 ml blood were taken and new sodium citrate syringes been placed on the catheter lines. Betaine solution A of example 2, at 40 mg/ml was subcutaneously administrated (15 mg/kg) to rats. At T0+50′ and T0+70′ the sampling operations of 1.5 ml bloods were repeated, followed by rats' euthanasia. Blood samples were centrifuged at 4000 tours/min during 20 min as to obtain Poor Platelet Plasma (PPP). As previously discussed ULMWH was considered to have Anti-Xa activity of ±150 IU/mg. Anti-Xa activities were determined using a chromogenic assay and Automatic Coagulation Laboratory ACL 200® (Instrumentation Laboratory, France).

| | Timing | | | | |
|---|---|---|---|---|---|
| | T0 | T0 + 30′ | T0 + 30′ | T0 + 50′ | T0 + 70′ |
| Treatmen | ULMWH | Blood | Betaine | Blood | Blood |
| | 5 mg/kg IV | sample | 15 mg/kg | sample | sample |
| | | | sc | | |

Results

| | Anti-Xa U/ml | | |
|---|---|---|---|
| | Sample 30′ | Sample 50′ | Sample 70′ |
| Rat # 1 | 1.47 | 1.05 | 0.44 |
| Rat # 2 | 1.51 | 1.11 | 0.61 |
| Rat # 3 | 1.37 | 0.88 | 0.53 |
| Rat # 4 | 1.95 | 1.14 | 0.67 |
| Rat # 5 | 1.67 | 1.08 | 0.54 |
| Rat # 6 | 1.77 | 0.95 | 0.47 |
| Rat # 7 | 1.67 | 0.77 | 0.66 |
| Rat # 8 | 1.86 | 1.21 | 0.84 |
| Rat # 9 | 1.87 | 1.33 | 0.65 |
| Rat # 10 | 1.35 | 0.93 | 0.44 |
| Rat # 11 | 2.01 | 1.43 | 0.91 |
| Mean | 1.68 | 1.08 | 0.61 |
| S. Deviation | ±0.23 | ±0.20 | ±0.16 |

Comments

At T0+70′ the iso-osmotic solution of Betaine at 40 mg/ml solution reduced ULMWH Anti-Xa activity by 63.7%. This reduction occurred uniformly in all animals.

This study was designed to avoid as much as possible bias and artefacts, each animal being its own control. Results show that Betaine has deep effects on ULMWH anticoagulation reversing

### Example 9

In Vitro Neutralization of Arixtra® and Lovenox® with Betaine on Human Plasma

OBJECTIVE: in vitro neutralization of Arixtra and Enoxaparin anti Xa activities with Betaine.

Materials and Methods:

Materials:

1. Arixtrag, fondaparinux sodium

2. Enoxaparin®, enoxaparin sodium

3. Betaine

4. Blood Bank Plasma

5. PT reagent (Innovin)

6. APTT reagents (Org Tek)

7. Thrombin for AIIa

8. Chromogenic substrate for AIIa

9. Bovine Factor Xa

10. Chromogenic substrate AXa.

11. ACL300+.

Methods:

Arixtra and Enoxaparin are made at a stock concentration of 100 μg/ml. Betaine is made at a stock concentrations of 10 mg/ml. Blood Bank Plasma is thawed. Arixtra and Enoxaparin are supplemented in the plasma in the concentration range of 1.25 μg/ml to 10 μg/ml. One ml of each of these concentrations is made. From these a separate set containing 0.5 ml of each of these concentrations is made. Betaine 0.5 ml at 10 mg/ml is added to each of these concentrations and also a control. Assays such as PT, APTT, AXa and AIa were performed on ACL300+.

Results & Observations:

In the AXa assay, Arixtra at concentrations of 10. 5. 2.5 and 1.25 μg/ml gave about 98% inhibition of Xa activity. After adding Betaine at a concentration of 5 mg/ml to each of these concentrations brought down the percentage of AXa inhibition by about 25% at 10 μg/ml and about 38% at 1.25 μg/ml. With Enoxaparin, the percentages inhibition of AXa activity at 10. 5. 2.5 and 1.25 μg/ml were 91.1, 80.8, 65.7 and 44.6%. The corresponding values after addition of Betaine were 42.1, 27.1, 4.9 and 0 giving neutralization for up to 53. 67. 92 and 100% respectively.

Even though Arixtra does not have any anti-IIa activity, but yet it was tested to confirm. No anti-IIa activity was noticed. However, with Enoxaparin the anti-IIa activities at 10, 5, 2.5 and 1.25 μg/ml were 63.5, 38.0, 11.6 and 0 respectively. After the addition of Betaine the corresponding activities were 9.6, 0. 0 and 0 respectively. The percent neutralizations with Betaine at the above concentrations were 84, 100, 100, and 0%. It is important to note that besides the percent neutralization of Xa activity there is also neutralization of the Ia activity with Enoxaparin.

Usually the anti-IIa activity reflects the hemorrhagic potential of a drug and this being neutralized with Betaine, it seems that the hemorrhagic potential of Enoxaparin could be avoided using Betaine. So it is important to note both the inhibition of the Xa and IIa activities while a synergistic effect on antithrombotic effect is seen through clotting assays. This is quite promising in clinical practice betaine avoiding hemorrhagic effects while retaining and potentialising the antithrombotics effects of the two molecules, namely enoxaparin sodium and fondaparinux sodium. A pharmaceutical combination with a compound of formula (CH₃)₃N⁺(CH₂)ₙCOO⁻ with n an integer from 1 to 5, preferably glycine betaine or a pharmaceutically acceptable salt thereof, esters thereof, precursors thereof, and mixtures thereof and enoxaparin sodium and/or fondaparinux sodium seems quite promising, showing improved therapeutically effects while possessing a safer profile regarding side effects, among them bleeding.

The percentage of Arixtra anti-Xa inhibition by Betaine was appreciatively 25% at 10 μg/ml and 38% at 1.25 μg/ml, the lowest concentration representing almost 4 times the 0.34 μg/ml concentration usually found in human during clinical approval trials. These results clearly mean that at fondaparinux clinical concentrations of 0.34 μg/ml, betaine solution will be able to reverse more Arixtra's anti-Xa activity.

At the same time on human plasma betaine solution almost completely neutralized Lovenox® anti-Xa (67. 92 & 100%) and anti-IIa (84. 100 & 100%) activities at enoxaparin doses respectively of 10. 5 & 2.5 μg/ml. Thus, safe and efficient antidotes as betaine(s) can allow enoxaparin sodium extended uses, as for example in CPB, coronary artery bypass graft surgery (CABG), haemodialysis, extracorporeal circulation, angioplasty, stents placements, prostheses placements, orthopaedic surgery, transplantation surgery or in general surgery.

In one embodiment of the invention a Kit containing both fondaparinux and a betaine is described.

In one embodiment of the invention a Kit containing both Idraparinux and a betaine is described.

Results

| Samples | Conc | ODAXa | % Inh Axa | ODAIIa | % IAIIa | PT | APTT |
|---|---|---|---|---|---|---|---|
| Arixtra 10 μg | 10 μg/ml | 0.017 | 98.7 | 0.869 | 0.0 | 9.3 | 40.9 |
| 5 | | 0.018 | 98.7 | 0.852 | 0.0 | 9.15 | 36.7 |
| 2.5 | | 0.02 | 98.5 | 0.872 | 0.0 | 9 | 33.9 |
| 1.25 | | 0.036 | 97.3 | 0.883 | 0.0 | 8.7 | 32.4 |
| Arixtra 10 μg | Bet 5 mg/ml | 0.353 | 73.5 | 0.978 | 0.0 | 10.6 | 65.9 |
| 5 | ″ | 0.253 | 81.0 | 0.981 | 0.0 | 10.5 | 61.2 |
| 2.5 | ″ | 0.357 | 73.2 | 0.998 | 0.0 | 10.5 | 56.7 |
| 1.25 | ″ | 0.54 | 59.5 | 1.013 | 0.0 | 10.3 | 54.4 |
| Enox 10 μg | 10 μg/ml | 0.119 | 91.1 | 0.293 | 63.5 | 8.7 | 62.9 |
| 5 | | 0.256 | 80.8 | 0.497 | 38.0 | 8.25 | 43.4 |
| 2.5 | | 0.457 | 65.7 | 0.709 | 11.6 | 8.25 | 35.7 |
| 1.25 | | 0.739 | 44.6 | 0.802 | 0.0 | 8.25 | 31.2 |
| Enox 10 μg | Bet 5 mg/ml | 0.772 | 42.1 | 0.725 | 9.6 | 11.4 | 113 |
| 5 | ″ | 0.973 | 27.1 | 0.851 | 0.0 | 10.2 | 73.2 |
| 2.5 | ″ | 1.268 | 4.9 | 0.923 | 0.0 | 10.2 | 58.2 |
| 1.25 | ″ | 1.563 | 0.0 | 0.966 | 0.0 | 10.2 | 51.2 |
| 0 | 0 | 1.334 | 0 | 0.802 | 0 | 8.25 | 26.2 |
| 0 | Betaine | 0 | 0 | 0 | 0 | 9.6 | 41.4 |

### Example 10

Realization of a Unit Oral Dosage Form Sachet Containing Betaine

Sachets material: Clay coated Paper 50 g/PR 12 g/Alufoil 7 my/Co+PE 5 g+18 g Technical specifications of the coating or primary packaging material:

| Substance | | g/m² | ASTM D 646 | 104 |
|---|---|---|---|---|
| Yield | | m²/kg | | 9.6 |
| Thickness | | mm | DIN 53105 | 0.09 |
| Permeability | | | | |
| Water Vapor (25° C.) | | g/m² · 24 h | DIN 53122 T2 | 0 |
| Oxygen (23° C./50% r.f.) | | ml/m² · 24 h · Atm | DIN 53380 T3 | 0 |
| Tensile strength | MD | KN/m | DIN 53455 | 4.8 |
| | CD | | | 2.8 |

| | | | |
|---|---|---|---|
| Coefficient of friction | outside/outside | DIN 53375 | 0.3 |
| COF | inside/inside | | 0.5 |
| Melting point of sealing | ° C. | | 105 |
| Material | | | |
| Max. Temp. of sealing | ° C. | | 200 |
| jaws | | | |

Batches of Betaine anhydrous and/or Betaine monohydrate and/or theirs mixtures having a controlled and defined initial level of moisture were tested with the processes of the invention as to control their biological activity. Then the controlled batches were packaged as unit oral dosage forms of 2000 mg of betaine using as primary packaging material multilayer Alufoil material. The dimensions of the sachets were 65 mm×90 mm. The realized sachets were weighted just after their manufacture and 6 months later, so as to determine the possible water intake of the betaine. The results show that the weight variation was in accordance with the invention and in the limits allowed by the International Pharmacopoeia (FDA/EMEA) for this sort (sachets/blister/pouches) of pharmaceutical dosage form, thus meaning also that the betaine inside the sealed sachets was not submitted to deliquescence or water intake. Thus such sachets with particular MVTR permit the betaines preservation for long periods. Optionally other drugs such as aspirin, anti cholesterol agents, hypertension drugs, anti diabetic drugs, anti-inflammatory drugs, anti-cancerous drugs, antioxidants, bioflavonoids, Ginkgo biloba, veinotonics and theirs mixtures can be added with betaines in the sachets.

### Example 11

Three different betaines coming from different suppliers, but all claiming pharmaceutical grade and claiming purity superior to 99% were tested according to the methods of the invention as described in example 3 and in claim 1.

Results

| | |
|---|---|
| Azure A + Heparin UFH | 0.449 |
| Azure A + Betaine 1 + Heparin UFH | 0.957 |
| Azure A + Betaine 2 + Heparin UFH | 0.704 |
| Azure A + Betaine 3 + Heparin UFH | 0.775 |

The 3 betaines where then assayed in The Laboratory of Chemical Pharmacy of the University of Liege. Infrared spectrometry as Magnetic Resonance (BRUKER® apparatus) did not show any difference in the profile of the 3 betaines.

The elementary analysis performed in duplicate in the same laboratory gave the following results:

% in theory: N, 11.96%; C, 51.26%; H, 9.46%

Betaine 1

% found (1): N, 11.65%; C, 50.38%; H, 9.73%;

% found (2): N, 11.67%; C, 50.50%; H, 9.52%;

Betaine 2

% found (1): N, 11.50%; C, 49.65%; H, 9.50%;

% found (2): N, 11.47%; C, 49.54%; H, 9.76%;

Betaine 3

% found (1): N, 11.68%; C, 51.02%; H, 9.44%;

% found (2): N, 11.66%; C, 50.86%; H, 9.36%;

The in vivo tests performed on animal as in example 5 showed a significant higher efficiency of Betaine 1 comparatively to betaine 2 and betaine 3. The whole blood aggregation tests also showed a significant higher efficiency of Betaine 1 comparatively to the 2 others.

For their in vivo efficiency the betaines can be ranked as follow:

Betaine 1>>Betaine 3>Betaine 2

Thus, the tests of the invention provide a reliable method to select the betaines with the most interesting pharmacological properties, when others tests are silent or enable to detect such differences. Accordingly, processes of betaine production comprising one or more Azure A tests of the invention are claimed.

Accordingly, processes of production of medicaments or drugs containing betaine as therapeutically effective ingredient are claimed. Said processes of production of medicaments comprising one or more Azure A tests of the invention.

What I claim is:

1. A pharmaceutical composition containing a therapeutic active amount of glycine betaine, whereby the glycine betaine has the property characterized in that when combining 0.4 ml of a mixed aqueous solution containing both unfractionned heparin in a final concentration of 6000 IU L⁻¹ and the glycine betaine of said pharmaceutical composition in a final glycine betaine concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

2. The composition of claim 1, which is an aqueous solution having a pH from 5.0 to 8.0 with a betaine concentration of from 5 to 500 mg/ml.

3. The composition of claim 1, which is a sterile and pyrogen-free pharmaceutical injectable solution

4. A pharmaceutical composition containing a therapeutic active amount of glycine betaine, whereby the glycine betaine has a pharmacological activity characterized in that when combining 0.4 ml of a mixed aqueous solution containing both unfractionned heparin in a final concentration of 6000 IU L⁻¹ and the glycine betaine of said pharmaceutical composition in a final glycine betaine concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

5. The composition of claim 1, whereby the glycine betaine has the property that when preparing at 20° C. a betaine solution by combining 0.4 ml of a mixed aqueous solution containing both unfractionned heparin in a final concentration of 6000 IU L⁻¹ and glycine betaine of said pharmaceutical composition in a final glycine betaine concentration of 10 mg L⁻¹ with 4 ml solution of azure A at 4×10⁻⁵ mol L⁻¹ said betaine solution with a total volume of 4.4 ml, after being mixed and at a temperature of 20° C., has a spectrometric absorbance of at least 0.95 at 632 nm wave length.

6. The composition of claim 1, whereby the glycine betaine has the property that when preparing at 20° C. a betaine solution by combining 0.4 ml of a mixed aqueous solution containing both unfractionned heparin in a final concentration of 6000 IU L⁻¹ and glycine betaine of said pharmaceutical composition in a final glycine betaine concentration of 10 mg L⁻¹ with 4 ml solution of azure A at 4×10⁻⁵ mol L⁻¹ said betaine solution with a total volume of 4.4 ml, after being mixed and at a temperature of 20° C., has a spectrometric absorbance of at least 1.0 at 632 nm wave length.

7. The composition of claim 1, whereby the glycine betaine has the property that when preparing at 20° C. a betaine solution by combining 0.4 ml of a mixed aqueous solution containing both unfractionned heparin in a final concentration of 12,000 IU L⁻¹ and glycine betaine of said pharmaceutical composition in a final glycine betaine concentration of 10 mg L⁻¹ with 4 ml solution of azure A at 4×10⁻⁵ mol L⁻¹, said betaine solution with a total volume of 4.4 ml, after being mixed and at a temperature of 20° C., has a spectrometric absorbance of at least 0.9 at 632 nm wave length.

8. The composition of claim 1, which further comprises at least one physiologically acceptable salt additive selected from the group consisting of sodium chloride, sodium hydroxide, sodium sulfate, magnesium chloride, magnesium hydroxide, magnesium sulfate, potassium chloride, potassium hydroxide, potassium sulfate and mixtures thereof.

9. The composition of claim 1, in which the glycine betaine has a purity of more than 99.5%.

10. The composition of claim 1, which is contained in a sterilized sealed container.

11. The composition of claim 1 which is contained in a sealed container, in which the sealed container has a layer in contact with the composition which is made of a synthetic material selected from the group consisting of polyethylene, polypropylene, copolymers of ethylene and propylene, polycarbonate, and mixtures thereof.

12. The composition of claim 10 which is contained in a sealed container, in which the sealed container has a layer forming a barrier to the light.

13. The composition of claim 1 which is an aqueous pharmaceutical composition containing a therapeutic active amount of glycine betaine, whereby the glycine betaine has the property that when preparing at 20° C. a betaine solution by combining 0.4 ml of a mixed aqueous solution containing both unfractionned heparin in a final concentration of 6000 IU L⁻¹ and glycine betaine of said pharmaceutical composition in a final glycine betaine concentration of 10 mg L⁻¹ with 4 ml solution of azure A at 4×10⁻⁵ mol L⁻¹, said betaine solution with a total volume of 4.4 ml, after being mixed and at a temperature of 20° C., has a spectrometric absorbance of at least 0.9 at 632 nm wave length.

14. The composition of claim 1, which is an aqueous composition having an osmolality at 20° C. comprised between 250 and 1250 mOsm/kg.

15. The composition of claim 1, which is an aqueous composition whereby at least 95% by weight of the composition consists of:
physiologically acceptable compound selected from the group consisting of glycine betaine, physiologically acceptable salts thereof and mixtures thereof, and
physiologically acceptable solvent.

16. The composition of claim 1, which is an aqueous composition whereby at least 99% by weight of the composition consists of:
physiologically acceptable compound selected from the group consisting of glycine betaine, physiologically acceptable salts thereof and mixtures thereof, and
physiologically acceptable solvent.

17. The composition of claim 1, which is an aqueous composition which has a viscosity comprised between 0.5 and 50 m Pa·s.

18. The composition of claim 1, which is an aqueous purified composition which has been submitted to a purifying step selected from the group consisting of microfiltration, ultrafiltration, nanofiltration, a reverse osmosis and mixtures thereof.

19. The composition of claim 1, which is pyrogens, endotoxins, pesticides, herbicides and heavy metals free.

20. The composition of claim 1, which is a composition submitted to at least one process selected from the group consisting of autoclaving process, pasteurization process, sterilisation process and combinations thereof.

21. The composition of claim 1, which is a composition submitted to at least one process selected from the group consisting of autoclaving process, pasteurization process, sterilisation process and combinations thereof after conditioning the composition in sealed containers.

22. The composition of claim 1, said composition being contained in a sealed container and being sterilized in the sealed container.

23. The composition of claim 1, which is an aqueous purified composition which has been submitted to a purifying step selected from the group consisting of microfiltration, ultrafiltration, nanofiltration, reverse osmosis and mixtures thereof.

24. The composition of claim 1, which is an aqueous composition submitted to a filtration with an absolute filter of less than 0.3 μm.

25. The composition of claim 1, which is an aqueous composition submitted to a filtration with a filter with opening with a diameter equal or lower than 0.1 μm.

26. The composition of claim 1, which is contained in a sealed container having at least one inner surface not in contact with the solution, whereby said inner surface is substantially free from salt deposits.

27. The composition of claim 1, which comprises a physiologically acceptable solvent selected from the group consisting of water, ethanol, polyethylene glycol, dimethylacetamide, aqueous polyvinylpyrrolidone, propylene glycol and mixtures thereof.

28. The composition of claim 1, which comprises only pyrogen free water as physiologically acceptable solvent.

29. The composition of claim 1, which is a solution whereby at least 95% by weight of the solution consists of:
physiologically acceptable compound selected from the group consisting of glycine betaine, physiologically acceptable salts thereof and mixtures thereof;
at least one physiologically acceptable salts of only one element selected from the group consisting of sodium, magnesium and potassium; and
a physiologically acceptable solvent.

30. The composition of claim 1, which is a solution in which the concentration of betaine is from 25 to 500 mg/ml.

31. The composition of claim 1 which further comprises a tonicity adjusting agent.

32. A process for the preparation of an aqueous pharmaceutical composition containing a therapeutic active amount of glycine betaine, whereby the glycine betaine has the property that when preparing at 20° C. a betaine solution by combining 0.4 ml of a mixed aqueous solution containing both unfractionned heparin in a final concentration of 6000 IU L⁻¹ and glycine betaine of said pharmaceutical composition in a final glycine betaine concentration of 10 mg L⁻¹ with 4 ml solution of azure A at 4×10⁻⁵ mol L⁻, said betaine solution with a total volume of 4.4 ml, after being mixed and at a temperature of 20° C., has a spectrometric absorbance of at least 0.9 at 632 nm wave length, said process comprising at least the following steps:
preparation of a solution comprising betaine or a salt thereof, said solution being enriched in sodium or magnesium or potassium,
filtration of the solution,
filling of vials,
sealing of the filled vials, and
sterilisation of the sealed vials,
whereby at least at one step selected from the group consisting of before the preparation of the solution comprising betaine, after the preparation of the solution comprising betaine, before the sterilisation, after the sterilisation, before the filling of vials, after the filling of vials, and combinations thereof, the activity of the glycine betaine is determined in order to confirm a pharmacological activity characterized in that when combining and mixing at 20° C. 0.4 ml of a mixed aqueous solution containing both unfractionned heparin in a final concentration of 6000 IU L⁻¹ and glycine betaine in a final concentration of 10 mg L⁻¹ with 4 ml solution of azure A at 4×10⁻⁵ mol L⁻¹ said 4.4 ml of combined and mixed aqueous solution has a spectrometric absorbance of at least 0.9 at 632 nm wave length at a temperature of 20° C.

33. The process of claim 32, whereby at least at one step, the pharmacological activity of the composition of at least one sealed vial is determined in order to confirm a pharmacological activity characterized in that when combining and mixing at 20° C. 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined and mixed aqueous solution has a spectrometric absorbance of at least 0.95 at 632 nm wave length at 20° C.

34. The process of claim 32, whereby at least at one step, the pharmacological activity of the composition of at least one sealed vial is determined in order to confirm a pharmacological activity characterized in that when combining and mixing at 20° C. 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined and mixed aqueous solution has a spectrometric absorbance of at least 1.0 at 632 nm wave length at 20° C.

35. The process of claim 32, whereby at least before the preparation of the solution comprising betaine, the activity of the glycine betaine is determined in order to confirm a pharmacological activity characterized in that when combining and mixing at 20° C. 0.4 ml of a mixed aqueous solution containing both unfractionned heparin in a final concentration of 12,000 IU L⁻¹ and glycine betaine in a final concentration of 10 mg L⁻¹ with 4 ml solution of azure A at 4×10⁻⁵ mol L⁻¹ said 4.4 ml of combined and mixed aqueous solution has a spectrometric absorbance of at least 0.9 at 632 nm wave length at 20° C.

36. The process of claim 32, whereby at least after the preparation of the solution comprising betaine or a salt thereof, the activity of the composition of at least one sealed vial is determined in order to confirm a pharmacological activity characterized in that when combining and mixing at 20° C. 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined and mixed aqueous solution has a spectrometric absorbance of at least 0.95 at 632 nm wave length at 20° C.

37. The process of claim 32, whereby at least after the preparation of the solution comprising betaine or a salt thereof, the activity of the composition of at least one sealed vial is determined in order to confirm a pharmacological activity characterized in that when combining and mixing at 20° C. 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined and mixed aqueous solution has a spectrometric absorbance of at least 1.0 at 632 nm wave length at 20° C.

38. The process of claim 32, whereby at least after the preparation of the solution comprising betaine or a salt thereof, the activity of the composition of at least one sealed vial is determined in order to confirm a pharmacological activity characterized in that when combining and mixing at 20° C. 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined and mixed aqueous solution has a spectrometric absorbance of at least 1.2 at 632 nm wave length at 20° C.

39. The process of claim 32, whereby at least after the preparation of the solution comprising betaine or a salt thereof, the activity of the composition of at least one sealed vial is determined in order to confirm a pharmacological activity characterized in that when combining and mixing at 20° C. 0.4 ml of a mixed aqueous solution containing both unfractionned heparin in a final concentration of 12,000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined and mixed aqueous solution has a spectrometric absorbance of at least 0.9 at 632 nm wave length at 20° C.

40. Process for selecting highly pharmacological active betaine in which the betaine is selected if said betaine has the property characterized in that when combining 0.4 ml of a mixed aqueous solution containing both unfractionned heparin in a final concentration of 6000 IU L⁻¹ and said betaine in a final concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

41. The process of claim 40 in which the combined aqueous solution is submitted to a spectrometric absorbance test at 20° C. and at 632 nm wave length, whereby in case a spectrometric absorbance of at least 0.95 at 632 nm wave length at a temperature of 20° C. is measured, the betaine is selected.

42. The process of claim 40 in which the combined aqueous solution is submitted to a spectrometric absorbance test at 20° C. and at 632 nm wave length, whereby in case a spectrometric absorbance of at least 1.0 at 632 nm wave length at a temperature of 20° C. is measured, the betaine is selected.

43. The process of claim 40 in which the combined aqueous solution is submitted to a spectrometric absorbance test at 20° C. and at 632 nm wave length, whereby in case a spectrometric absorbance of at least 1.2 at 632 nm wave length at a temperature of 20° C. is measured, the betaine is selected.

44. The process of claim 40 whereby the betaine is selected if said betaine has the property characterized in that when combining 0.4 ml of a mixed aqueous solution containing both unfractionned heparin in a final concentration of 12000 IU L⁻¹ and said betaine in a final concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

45. A method for treating at least one side effect of a compound selected from the group consisting of heparins and heparin like compounds, synthetic heparins, synthetic heparin like compounds, low molecular heparins, ultra low molecular heparins and pentasacharides, Arixtra, Idraparinux sodium, ultra low molecular heparins, directs and indirects anti-Xa agents such as DX 9065a, anti factor IX agents, anti factor VII agents, directs and indirects anti coagulation factor agents, anti-IIa agents such as argatroban, ximelagatran, Exanta, melagatran, lepirudin, desirudin, recombinant hirudins and hirulog, direct thrombin inhibitors, hirudin, bivalirudin, Angiomax, argatroban, efegatran, inogatran and compounds structurally similar to the preceding compounds as to prevent at least one hemorrhagic side effect and/or for potentialising at least one therapeutically effect of one or more compounds selected from the above compounds and mixture thereof, administered to a human at a dose considered as safe for ensuring an anticoagulation effect, said method consisting of administering a pharmaceutical composition containing a therapeutic active amount of glycine betaine, whereby the glycine betaine has a pharmacological activity characterized in that when combining 0.4 ml of a mixed aqueous solution containing both unfractionned heparin in a final concentration of 6000 IU L⁻¹ and the glycine betaine of said pharmaceutical composition in a final glycine betaine concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

46. The method of claim 45, in which the side effect to be treated is selected from the group consisting of thrombocytopenia, hemorrhagic side effect, bleeding side effect and combination thereof.

47. The method of claim 45, in which the glycine betaine is administered as a betaine sterile and pyrogen-free physiologically acceptable pharmaceutical injectable composition having a pH adjusted from 5.0 to 8.0 with a betaine pharmacological activity characterized in that when combining 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

48. The method of claim 45, in which the glycine betaine is administered as a betaine sterile and pyrogen-free physiologically acceptable pharmaceutical injectable composition having a pH adjusted to from 5.0 to 8.0 with a betaine concentration of from 10 to 500 mg/ml, wherein said solution has an osmolality comprised between 250 and 1450 mOsm/kg, whereby the composition has such an activity characterized in that when combining 0.4 ml of a mixed aqueous solution containing both unfractionned heparin in a final concentration of 6000 IU L⁻¹ and glycine betaine of said pharmaceutical composition in a final concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

49. A method for treating one or more trouble selected from the group of lupus anticoagulant, miscarriage, pregnancy, antiphospholipid syndrome, thrombotic and/or obstetric complications, specially miscarriages and/or repeated fetal deaths, pregnancy troubles, intra- and postpartum, hemorrhoids anticardiolipin antibodies, primary and/or secondary haemostatic disorders, prevention in travel induced thrombosis such as air travel deep venous thrombosis, chronic venous insufficiency CVI grade I or II Widmer classification, heavy legs, portal hemorrhage, portal hypertension, pulmonary hypertension, bleeding of oesophageal varices, sepsis and severe sepsis, coagulopathy, disseminated intravascular coagulation (DIC), complications in sepsis, polyps, nasal polyps, scleroderma, malaria, uncontrolled cascade of coagulation, fibrinolysis, inflammation, Nash & liver diseases, homocystinuria, homocysteinemia, sepsis, septic shocks, bleeding, hypertension, Alzheimer disease, vascular dementia, digital ischemia, Raynaud's Phenomenon, pulmonary hypertension, intermittent claudication, degenerative diseases, portal hypertension, hypertension, vascular hypertension, ocular hypertension, gangrene, diabetes, cardiovascular diseases, cerebrovascular diseases, peripheral arterial diseases, heart diseases, angina pectoris, atrial fibrillation, inflammation diseases, kidney diseases, cancer diseases, sexual dysfunction and metabolic syndrome said method consisting of administering a pharmaceutical composition containing a therapeutic active amount of glycine betaine, whereby the glycine betaine has a pharmacological activity characterized in that when combining 0.4 ml of a mixed aqueous solution containing both unfractionned heparin in a final concentration of 6000 IU L⁻¹ and the glycine betaine of said pharmaceutical composition in a final glycine betaine concentration of 10 mg L⁻¹ with 4 ml solution of azure A at 4×10⁻⁵ mol L⁻¹ said 4.4 ml of combined aqueous solutions have a spectrometric absorbance of at least 0.9 at 632 nm wave length after been mixed at a temperature of 20° C.

50. An oral pharmaceutical composition containing a therapeutic active amount of glycine betaine, whereby the glycine betaine has the property that when preparing at 20° C. a betaine solution by combining 0.4 ml of a mixed aqueous solution containing both unfractionned heparin in a final concentration of 6000 IU L⁻¹ and glycine betaine of said pharmaceutical composition in a final glycine betaine concentration of 10 mg L⁻¹ with 4 ml solution of azure A at 4×10⁻⁵ mol L⁻¹, said betaine solution with a total volume of 4.4 ml, after being mixed and at a temperature of 20° C., has a spectrometric absorbance of at least 0.9 at 632 nm wave length.

51. The composition of claim 50, which is an aqueous purified composition which has been submitted to a purifying step selected from the group consisting of microfiltration, ultrafiltration, nanofiltration, reverse osmosis and mixtures thereof.

52. A pharmaceutical unit dosage form of a composition containing a therapeutic active amount of glycine betaine, whereby the glycine betaine has the property that when preparing at 20° C. a betaine solution by combining 0.4 ml of a mixed aqueous solution containing both unfractionned heparin in a final concentration of 6000 IU L⁻¹ and glycine betaine of said pharmaceutical composition in a final glycine betaine concentration of 10 mg L⁻¹ with 4 ml solution of azure A at 4×10⁻⁵ mol L⁻¹, said betaine solution with a total volume of 4.4 ml, after being mixed and at a temperature of 20° C., has a spectrometric absorbance of at least 0.9 at 632 nm wave length, said dosage form being selected from the group consisting of sachets, pouches, blisters and bags, wherein the pharmaceutical unit dosage form is provided with moisture barrier property defined by an increase of weight of the composition of less than 1% after storage of the unit dosage form in sealed condition in an environment with a temperature of 38° C. and a relative humidity of 90% during 30 days.

53. The composition of claim 50 wherein the composition is submitted to a drying process before being sealed in the pharmaceutical unit dosage form which is provided with a moisture barrier property defined by an increase of weight of the compositions of less than 1% after storage of the unit dosage form in sealed condition in an environment with a temperature of 38° C. and a relative humidity of 90% during 30 days.

54. The composition of claims 51 wherein the composition is submitted to a drying process before being sealed in the pharmaceutical unit dosage form which is provided with a moisture barrier property defined by an increase of weight of the compositions of less than 1% after storage of the unit dosage form in sealed condition in an environment with a temperature of 38° C. and a relative humidity of 90% during 30 days.

55. The composition of claims 52 wherein the composition is submitted to a drying process before being sealed in the pharmaceutical unit dosage form which is provided with a moisture barrier property defined by an increase of weight of the compositions of less than 1% after storage of the unit dosage form in sealed condition in an environment with a temperature of 38° C. and a relative humidity of 90% during 30 days.

56. A Pharmaceutical unit dosage form of a composition containing at least a betaine, said dosage form being selected from the group consisting of sachets, pouches, blisters and bags, wherein the pharmaceutical unit dosage form is provided with moisture barrier property defined by an increase of weight of the composition of less than 1% after storage of the unit dosage form in sealed condition in an environment with a temperature of 38° C. and a relative humidity of 90% for 30 days at a temperature of 38° C. and at 90% relative humidity during 24 hours.

57. A pharmaceutical unit dosage form of a composition containing at least a betaine, said dosage form being selected from the group consisting of sachets, pouches, blisters and bags, wherein the pharmaceutical unit dosage form is provided with moisture barrier property defined by an MVTR value inferior to 0.2 g/m² at a temperature of 38° C. and at 90% relative humidity during 24 hours.

58. The unit dosage form of claim 56, wherein the pharmaceutical unit dosage form is provided with moisture barrier property defined by an MVTR value inferior to 0.1 g/m² at a temperature of 38° C. and at 90% relative humidity during 24 hours.

59. The unit dosage form of claim 56, wherein the pharmaceutical unit dosage form is provided with moisture barrier property defined by an MVTR value inferior to 0.01 g/m² at a temperature of 38° C. and at 90% relative humidity during 24 hours.

60. The unit dosage form of claim 56, wherein the pharmaceutical unit dosage form is provided with moisture barrier property defined by an MVTR value inferior to 0.001 g/m² at a temperature of 38° C. and at 90% relative humidity during 24 hours.

61. The unit dosage form of claim 56, wherein the pharmaceutical unit dosage form is provided with moisture barrier property defined by an MVTR value inferior to 0.0001 g/m² at a temperature of 38° C. and at 90% relative humidity during 24 hours.

62. The pharmaceutical unit dosage form of claim 56, wherein the tensile strength, the tearing strength and the coefficients of friction of the packaging material are selected as to augment the pharmaceutical unit dosage form compliance.

63. The pharmaceutical unit dosage form of claim 56, comprising a barrier selected from the group consisting of oxygen barrier, light barrier and combinations thereof.
